SEQUENCE LISTING (5 sequences)

SEQ ID NO: 1 - protein, 131 aa, Diaphorina citri
QSSSTTLAPTMAQKVNAVATKCKTELNSPPEAVALLGAKSLPTDEKQRCFLECVYKNLGIVKDNKFSPEGSKALAAQRFKGDELTKANKLVETCAKEAVVAAGNTEKCALGKSVRSCFVKNGEKIDFFPKA

SEQ ID NO: 2 - protein, 113 aa, Diaphorina citri
PQKKQAESAPESGILDNFDVDSVLNNGRVLKNYVKCVLNMGPCTAEGREMKRVLPDVLKTACGKCSEQHKERLRKVLLKLKNDPKLKEDYNKIIEKYDPKKEYVANLEKFLLS

SEQ ID NO: 3 - protein, 112 aa, Diaphorina citri
KPQTKKYTTKYDNINLEEIIHNDRLLNNYYECLIGTGNCTPDGAELKRLLPDALENECKACSEKQKEGSEVMIKYLIDNKPEMWAELEKKYDSTGSYRKKYAAEAEKRGLKV

SEQ ID NO: 4 - protein, 116 aa, Diaphorina citri
KPAGEKYTTKYDNVDLDQILRNDRLFNNYYNCLLDKGKCSPDGQELKNKLPDAIATECKSCSEKQKEGSKRIFKYLIDNKPEQWAELEKKFDPNGTYKAKYQKELSDLKAGKPVKI

SEQ ID NO: 5 - protein, 105 aa, Diaphorina citri
SPAYTTKYDNVDLDEIIANDRLFTNYYKCLMDTGACSPDGAELKKVLPDAIATECGGCSDKQKEGAKKIFKFLIEKKPEEWKALEGKYDPSGSYKAKYDAQLKAL

(19) United States
(12) Patent Application Publication
(10) Pub. No.: US 2013/0287727 A1
(43) Pub. Date: Oct. 31, 2013

(54) Psyllid Attractants and Their Uses

(71) Applicant: INSCENT, INC., Irvine, CA (US)

(72) Inventors: Daniel F. Woods, Irvine, CA (US); Spiros Dimitratos, Ontario, CA (US)

(73) Assignee: INSCENT, INC., Irvine, CA (US)

(21) Appl. No.: 13/870,782

(22) Filed: Apr. 25, 2013

Related U.S. Application Data
(60) Provisional application No. 61/638,426, filed on Apr. 25, 2012.

(51) Int. Cl. A01N 43/40; C07D 207/12; C07D 207/24; C07D 211/74; G06F 17/18; C07D 307/83; C07D 307/84; A01N 43/36; A01N 43/12; C07D 211/42; C07D 307/82

(52) U.S. Cl. CPC A01N 43/40; C07D 211/42; C07D 207/12; C07D 207/24; C07D 211/74; C07D 307/82; C07D 307/83; C07D 307/84; A01N 43/36; A01N 43/12; G06F 17/18
USPC 424/84; 546/216; 546/221; 548/541; 549/467; 549/466; 702/179

(57) ABSTRACT

The present specification discloses psyllid attractants, compositions comprising such attractants, lures, traps and other devices using such attractants, methods and uses to attract, capture and/or kill psyllids using such attractants, compositions and/or lures, traps and/or other devices, and methods and uses for monitoring a psyllid population using such attractants, compositions and/or lures, traps and/or other devices.

This patent application claims priority pursuant to 35 U.S.C. §119(e) to U.S. Provisional Patent Application 61/638,426, filed Apr. 25, 2012, which is hereby incorporated by reference in its entirety.

Portions of the work described herein were supported by Small Business Innovation Research (SBIR) grant number 1014283 awarded by the National Science Foundation (NSF). The government of the United States of America may retain certain rights to this patent.

### INTRODUCTION

Psyllids or “jumping plant lice” are small phytophagous, phloem feeding insects that are typically monophagous (feed on a single plant) or oligophagous (feed on a few related plants). Psyllids of economic importance include pests such as the carrot, potato, citrus and avocado psyllids. Some of the other plants adversely affected by psyllids include: pear, apple, apricot, pistachio, olive, gum trees, wattles, bay, persimmon, lillypilly or rose apple, Leucaena, Pittosporum, Sideroxylon, and Tabebuia.

The Asian citrus psyllid, Diaphorina citri, is an agricultural pest responsible for very serious damage to citrus crops around the world. Endemic to Asia, over the past 30 years psyllids have been accidentally spread throughout much of the world's citrus producing areas in Asia, Africa, the Indian subcontinent, the Arabian Peninsula, Brazil, Mexico, and the United States. It appears that psyllids can spread not only from shipments of infected citrus or root stocks, but also from related host plants on which the insect can feed, such as, e.g., orange jasmine. Particularly alarming is the ability of psyllids to survive and propagate using host plants either in lieu of or in addition to agricultural citrus, and also its ability to spread to a variety of geographic locations, each with its own particular climate, geography, and flora.

Psyllids damage citrus crops in two ways. First, psyllid feeding on citrus plants results in ceased terminal elongation and malformed leaves and shoots, and the insects' excretions encourage mold growth. Both the arrested growth and mold infestation weaken the plants causing reduced fruit production and even death. However, by far the most significant damage caused by ACP is as a vector in the transmission and propagation of citrus greening disease (Huanglongbing or HLB). Nearly all citrus species, cultivars and hybrids are susceptible to HLB and the disease devastates affected groves, causing rapid tree decline. This disease causes chlorosis with yellow shoots, sparse foliage, leaf mottling, stunted tree growth, foliage loss and fruit loss. Infected trees produce smaller, abnormally shaped and colored, harder, and bitter fruit. When psyllids are abundant and environmental conditions are favorable, HLB can rapidly spread to existing trees, both commercial and residential. For example, young trees infected with HLB never come into fruit production, with most dying within two years from this disease. Infected mature trees decline in health and become non-productive within 5-8 years. In addition, ornamental trees which become infected with HLB become stunted and sparsely foliated, making them aesthetically displeasing for the home landscape. Thus, production costs in affected areas are severely increased since infected trees soon become non-productive and ultimately require replacement. HLB is thus a very serious disease that can truly limit citrus production.

Currently, HLB control comprises the replacement of infected cultures with clean stock, the thorough elimination of any sources of inoculum, fastidious control of psyllid populations on non-crop species in order to avoid disease vector reservoirs, and judicious use of pesticides to control psyllid populations on crop cultures. Unfortunately, these control measures are arduous to implement and manage and continued production of citrus has proven difficult and expensive in areas where it is widespread. In addition, despite all these efforts, nowhere in the world has HLB been successfully managed, much less eradicated.

One key limitation in controlling HLB is the lack of effective traps to monitor the extent and density of psyllid infestation and/or control ACP populations. Current trapping methods are basically “blunder” traps; minimally effective yellow sticky cards that rely purely on random collision events where the insect simply flies into the sticky surface of the trap. Although blunder traps with a color cue exist, there does not appear to be any appreciable difference in their ability to attract more insects. To date, there are no traps comprising attractant that effective lures psyllids onto the trapping surface.

The present specification discloses effective, efficient psyllid attractants for use as a lure in devices such as, e.g., traps, capture, or other devices. These compounds appear to activate the chemosensory system in psyllids thus invoking a behavioral response which cause the insects to move towards these attractants. When placed in or near an orchard, these traps essentially “compete” for psyllids with plant species upon which the insect relies for its survival. The disclosed devices comprising a psyllid attractant would benefit the citrus industry since it would yield data useful in monitoring the psyllid populations, in generating control strategies for reducing psyllid infestation, in biocontrol of invading plant species, in providing better control measures for managing, reducing, and eradicating HLB as well as other vectored diseases.

### SUMMARY

Aspects of the present specification disclose compounds that substantially mimic the attractant chemosensory cues of a natural compound produced by a citrus plant. The disclosed compounds are psyllid attractants and may having a binding affinity that is substantially the same as the binding affinity of the natural ligand for that psyllid OBP or SAP, wherein the compound is a psyllid attractant and/or have a dissociation equilibrium constant that is greater than the dissociation equilibrium constant of the natural ligand for that psyllid OBP or SAP. In some aspects, the disclosed compounds may substantially mimic the attractant chemosensory cues of (R)-(+)-Limonene and/or have a binding affinity that is substantially the same as the binding affinity of (R)-(+)-Limonene for that psyllid OBP or SAP and/or have a dissociation equilibrium constant that is greater than the dissociation equilibrium constant of (R)-(+)-Limonene for that psyllid OBP or SAP. In other aspects, the disclosed compounds may substantially mimic the attractant chemosensory cues of Petitgrain and/or have a binding affinity that is substantially the same as the binding affinity of Petitgrain for that psyllid OBP or SAP and/or have a dissociation equilibrium constant that is greater than the dissociation equilibrium constant of Petitgrain for that psyllid OBP or SAP.

Other aspects of the present specification disclose compounds having a structure of formula I:

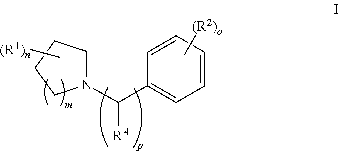

wherein R^{A} and R¹ are each independently R³, optionally substituted aryl, ═O, NR³R⁴, OR³, CN, NO₂, OCF₃, CF₃, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, OCF₃, CF₃, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl, OCF₃, or CF₃; m is 0, 1, 2, 3 or 4; n is 0, 1, 2, 3, 4, 5 or 6; p is 0, 1, 2, 3, 4, 5 or 6; and o is 0, 1, 2, 3, 4, or 5.

Other aspects of the present specification disclose compounds having a structure of formula X:

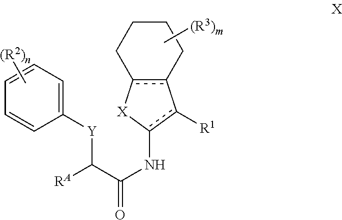

wherein a dashed line represent the presence or absence of a bond; R^{A}, R¹, R³ are each independently R⁴, optionally substituted aryl, ═O, NR⁴R⁵, OR⁴, CN, NO₂, OCF₃, CF₃, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; each R² is independently R⁴, optionally substituted aryl, NR⁴R⁵, OR³, CN, NO₂, OCF₃, CF₃, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; R⁴ and R⁵ are each independently H, optionally substituted alkyl, or CF₃; m is 0, 1, 2, 3, 4, or 5; n is 0, 1, 2, 3, 4, or 5; X is CHR^{A}, O, S, or NH; and Y is CHR^{A}, O, S, or NH.

Other aspects of the present specification disclose the following compounds: 1-(3,4-dichlorobenzyl)-3-piperidinol, ethyl 2-[(phenoxyacetyl)amino]-4,5,6,7-tetrahydro-1-benzothiophene-3-carboxylate, 2-{[(3-chloro-4-fluorophenyl)amino]methylene}-5,5-dimethyl-1,3-cyclohexanedione, 1-(4-chlorobenzyl)-4-(4-nitrophenyl)piperazine, dimethyl 2-(1,2,2-trimethyl-3-thioxo-2,3-dihydro-4(1H)-quinolinylidene)-1,3-dithiole-4,5-dicarboxylate, N,N′-(2-nitro-1,3-propanediylidene)dicyclohexanamine, 2-[2-(3,5-dimethyl-1H-pyrazol-1-yl)-6-phenyl-4-pyrimidinyl]phenol, 4-methyl-2-[2-nitro-5-(1-pyrrolidinyl)phenyl]-1(2H)-phthalazinone, 2-[(4-methylphenyl)amino]naphthoquinone, 3-ethyl-4,7-dimethoxy-2-methyl-1,3-benzothiazol-3-ium iodide, 4-hydroxy-3-[2-(2-hydroxy-5-methoxyphenyl)-2,3-dihydro-1,5-benzothiazepin-4-yl]-6-methyl-2H-pyran-2-one, 2,4-dibromo-6-[(1,3-dioxo-1,3-dihydro-2H-inden-2-ylidene)methyl]phenyl acetate, ethyl 2-[(2-hydroxy-5-methoxybenzylidene)amino]-4,5-dimethyl-3-thiophenecarboxylate, 2-[2-(4-chlorophenyl)vinyl]-3-phenyl-4(3H)-quinazolinone, 7-methyl-2-oxo-1,3-benzoxathiol-5-yl benzenesulfonate, N-(5-benzylidene-4-oxo-2-thioxo-1,3-thiazolidin-3-yl)-2-methoxybenzamide, 2-methyl-N′-(2-oxo-1,2-dihydro-3H-indol-3-ylidene)benzohydrazide, 5-(4-aminophenoxy)-2-phenyl-1H-isoindole-1,3(2H)-dione, 3-chloro-6-fluoro-N-(4-phenyl-1,3-thiazol-2-yl)-1-benzothiophene-2-carboxamide, N′-(2-hydroxy-5-methoxybenzylidene)-4-(2-oxo-1-pyrrolidinyl)benzohydrazide, 2-(2,5-dichlorophenyl)-1,3-benzoxazol-5-amine, 2-hydroxy-N′-[(2-hydroxy-1-naphthyl)methylene]-2-phenylpropanohydrazide, 2-(3-bromo-2-hydroxy-5-nitrobenzylidene)-1-benzothiophen-3(2H)-one, 4-[2-(1,3-benzodioxol-5-yl)vinyl]quinoline, ethyl 2-amino-4-(4-bromophenyl)-5-oxo-4H,5H-pyrano[3,2-c]chromene-3-carboxylate, (5-chloro-2-methylphenyl)[3-(4-nitrophenyl)-2-propen-1-ylidene]amine, 1-{[(4′-nitro-4-biphenyl)imino]methyl}-2-naphthol, N-[4-chloro-3-(trifluoromethyl)phenyl]-7-nitro-2,1,3-benzoxadiazol-4-amine, 3-allyl-2-(benzylthio)-5-ethyl-6-hydroxy-4(3H)-pyrimidinone, 1H-indole-2-carbaldehyde (1H-indol-2-ylmethylene)hydrazone, 1-(2-bromobenzoyl)-4-phenylpiperazine, (4-bromobenzylidene)(7-nitro-9H-fluoren-2-yl)amine, 3-(3-bromophenyl)-5-(2-furylmethylene)-2-thioxo-1,3-thiazolidin-4-one, 2,4-dibromo-6-[(5-methyl-1,3,4-thiadiazol-2-yl)amino]phenol, 5-{3-[4-(dimethylamino) phenyl]-2-propen-1-ylidene}-2-thioxo-1,3-thiazolidin-4-one, 6-[2-(2,4-dichloro-5-nitrophenyl)vinyl]-4-(trifluoromethyl)-2(1H)-pyrimidinone, 5-{[(3,5-dimethoxyphenyl)amino]methylene}-1-(3-methylphenyl)-2-thioxodihydro-4,6(1H,5H)-pyrimidinedione, N-[3-(3-bromophenyl)-2-propen-1-ylidene]-4-(4-morpholinyl)aniline, 5-[4-(diethylamino)benzylidene]-2-(4-morpholinyl)-1,3-thiazol-4(5H)-one, 3-bromo-N′-(3-oxo-3-phenyl-1-propen-1-yl)benzohydrazide, 1-{[5-(4-nitrophenyl)-2-furyl]methyl}-4-phenylpiperazine, 1-(4-ethylcyclohexyl)-4-(4-nitrophenyl)piperazine, 1-[3-(2-nitrophenyl)-2-propen-1-yl]-4-(3-phenyl-2-propen-1-yl)piperazine, 1-(4-nitrophenyl)-4-(4-pyridinylmethyl)piperazine, 1-(4-nitrophenyl)-4-(3-phenyl-2-propen-1-yl)piperazine, 6,8-dichloro-3-{[4-(2-methoxyphenyl)-1-piperazinyl]methyl}-4H-chromen-4-one, 1-(bicycle[2.2.1]hept-5-en-2-ylmethyl)-4-(4-nitrophenyl)piperazine, 1-[(5-methyl-2-thienyl)methyl]-4-(4-nitrophenyl)piperazine, N,N-diethyl-4-[(phenylimino)methyl]aniline, 1-[4-(benzyloxy)phenyl]-3-methyl-1H-pyrrole-2,5-dione, 1-(4-nitrophenyl)-1H-indole, 4-{[(3-bromophenyl)imino]methyl}-1,3-benzenediol, 1,2-dimethyl-3-[2-(4-nitrophenyl)-2-oxoethyl]-1H-3,1-benzimidazol-3-ium bromide, 3-methyl-2-[4-oxo-5-(2-oxo-1,2-dihydro-3H-indol-3-ylidene)-2-thioxo-1,3-thiazolidin-3-yl]butanoic acid, 4-bromo-N′-(2-oxo-1,2-dihydro-3H-indol-3-ylidene)benzohydrazide, N-[2-(2-naphthyl)-1,3-dioxo-2,3-dihydro-1H-isoindol-4-yl]acetamide, N′-(1,7-dimethyl-2-oxo-1,2-dihydro-3H-indol-3-ylidene)-2-(4-methoxyphenoxy)acetohydrazide, 5-[(2-ethoxy-1-naphthyl)methylene]-2,4,6(1H,3H,5H)-pyrimidinetrione, 2-(2,6-dibromo-4-methylphenoxy)-N′-(2-oxo-1,2-dihydro-3H-indol-3-ylidene)acetohydrazide, 4-{4-[(4-nitrophenyl)thio]phenyl}-4-azatricyclo[5.2.1.0˜2,6˜]dec-8-ene-3,5-dione, 1-(4-bromophenyl)-5-[4-(dimethylamino)benzylidene]-2,4,6(1H,3H,5H)-pyrimidinetrione, 1-(2-methoxyphenyl)-5-(3-phenyl-2-propen-1-ylidene)-2,4,6(1H,3H,5H)-pyrimidinetrione, N-benzyl-7-chloro-4-nitro-2,1,3-benzoxadiazol-5-amine, 2-[3-(4-hydroxyphenyl)acryloyl]-1H-indene-1,3(2H)-dione, N′-(3,5-dibromo-2-hydroxybenzylidene)-4-[(4-methylphenyl)amino]butanohydrazide, 3-amino-6-cyclopropyl-4-phenylthieno[2,3-b]pyridine-2-carboxylic acid, 1-(dichloroacetyl)-4-(4-nitrophenyl)piperazine, 1-(5-bromo-2-methoxybenzyl)-4-(4-nitrophenyl)piperazine, 3-(3-bromophenyl)-5-(4-hydroxy-3-methoxybenzylidene)-2-thioxo-1,3-thiazolidin-4-one, 11-chloro-12-nitro-2-phenyl-2,4,5,6,7,8-hexahydro-3H-azepino[1,2-a]pyrazolo[4,3-c]quinolin-3-one, 4-benzyl-1-[(5-nitro-2-thienyl)methyl]piperidine, 3-[(benzylimino)methyl]-1,2-benzenediol, 2-[(6-nitro-1,3-benzodioxol-5-yl)methyl]-2,3,4,9-tetrahydro-1H-beta-carboline, 4-[4-(dimethylamino)benzylidene]-2-(3-nitrophenyl)-1,3-oxazol-5(4H)-one, 4-(2-oxo-1-pyrrolidinyl)-N′-[1-(2-pyridinyl)ethylidene]benzohydrazide, 2-[4-(methylthio)benzyl]-2,3,4,9-tetrahydro-1H-beta-carboline, 4-(dimethylamino)benzaldehyde (2-nitrophenyl)hydrazone, (4-bromophenyl)(5-methoxy-2-methyl-1-benzofuran-3-ylmethanone, 4-[(cyclohexylamino)methylene]-5-methyl-2-phenyl-2,4-dihydro-3H-pyrazole-3-thione, 4-bromo-2-{[(5-tert-butyl-2-hydroxyphenyl)imino]methyl}-6-ethoxyphenol, N′-(4-chlorobenzylidene)-1-methyl-5-phenyl-1H-pyrazole-3-carbohydrazide, N′-{[2-(4-bromophenyl)-3-indolizinyl]methylene}isonicotinohydrazide, ethyl 2-amino-4-(3,4-dichlorophenyl)-5-oxo-4H,5H-pyrano[3,2-c]chromene-3-carboxylate, 1H-indene-1,2,3-trione 2-(phenylhydrazone), 2-[(1,3-benzothiazol-2-ylamino)methylene]-1-benzothiophen-3(2H)-one, N-[2-[4-(dimethylamino)phenyl]-1-(1-piperazinylcarbonyl)vinyl]benzamide, 5-(2-hydroxy-3-methoxybenzylidene)-2-thioxo-1,3-thiazolidin-4-one, 1-[(4-chloro-3-nitrophenyl)sulfonyl]-2-methylpiperidine, 2-(1H-benzimidazol-2-ylthio)-N-(4-methylphenyl)acetamide, 3-[2-(4-chlorophenyl)-1,3-thiazol-4-yl]-7-hydroxy-2H-chromen-2-one, 4,4′-[1,4-phenylenebis(nitrilomethylylidene)]diphenol, 3-chloro-1-(4-chlorophenyl)-4-(dimethylamino)-1H-pyrrole-2,5-dione, 1-(4-bromophenyl)-3-chloro-4-(1-pyrrolidinyl)-1H-pyrrole-2,5-dione, N-{5-[4-(dimethylamino)benzylidene]-4-oxo-2-thioxo-1,3-thiazolidin-3-yl}-2-hydroxybenzamide, 5-{[5-(4-bromophenyl)-2-furyl]methylene}-1,3-thiazolidine-2,4-dione, 2,4,5-trimethylbenzaldehyde (4-amino-5-ethyl-4H-1,2,4-triazol-3-yl)hydrazone, 1-(2-chlorophenyl)-5-{[(3-chlorophenyl)amino]methylene}-2-thioxodihydro-4,6(1H,5H)-pyrimidinedione, 9-(3,5-dichloro-4-hydroxyphenyl)-3,3,6,6-tetramethyl-3,4,5,6,7,9-hexahydro-1H-xanthene-1,8(2H)-dione, 5-{3-methoxy-4-[2-oxo-2-(1-piperidinyl)ethoxy]benzylidene}-2-thioxo-1,3-thiazolidin-4-one, 3-(4-biphenylyl)-5,6-dihydroimidazo[2,1-b][1,3]thiazole hydrobromide, 5-(4-ethylbenzylidene)-2-thioxo-1,3-thiazolidin-4-one, 4-bromo-N′-(3-oxo-3-phenyl-1-propen-1-yl)benzohydrazide, 1-(2-fluorophenyl)-4-{[5-(2-nitrophenyl)-2-furyl]methyl}piperazine, 1-[(5-methyl-2-furyl)methyl]-4-(4-nitrophenyl)piperazine, N-benzyl-N-(4-nitrobenzyl)ethanamine, 6,8-dichloro-3-[(4-methyl-1,4-diazepan-1-yl)methyl]-4H-chromen-4-one, 3-[(4-acetyl-1-piperazinyl)methyl]-6,8-dichloro-4H-chromen-4-one, 2-(4-butoxybenzyl)-6,7-dimethoxy-1,2,3,4-tetrahydroisoquinoline, 2-{[butyl(methyl)amino]methyl}-4-nitrophenol, 6-chloro-3-{[2-(hydroxymethyl)-1-piperidinyl]methyl}-4H-chromen-4-one, 6,8-dichloro-3-(4-thiomorpholinylmethyl)-4H-chromen-4-one, 1-[(4-phenyl-1-piperazinyl)methyl]-2-naphthol, or 2-(1-azepanylmethyl)-6-ethoxyphenol.

Other aspects of the present specification disclose compositions comprising one or more of the compounds disclosed herein. The compositions may optionally comprise an adhesive, an insecticide, a solvent, a wetting agent, an emulsifying agent, a carrier, a diluent, a dispersing agent and/or a compatible matrix. The disclosed compositions may be a controlled-release device. The disclosed compositions may be a vapor or gas composition, a liquid composition, a semi-solid composition, or a solid composition.

Other aspects of the present specification disclose devices comprising one or more of the compounds disclosed herein and/or one or more of the compositions disclosed herein. The disclosed devices are designed to be a lure and/or trap that can attract and/or capture psyllids. The disclosed devices may be made in a manner designed to keep captured psyllids alive or to kill captured psyllids. The disclosed devices may further comprises an adhesive and/or an insecticide. The disclosed devices may further comprise a one-way entrance operationally-connected to a chamber, wherein the one-way entrance enables psyllids to enter into the chamber, but will prevent the psyllids from leaving the chamber once entered.

Other aspects of the present specification disclose methods of capturing psyllids. The disclosed methods may comprises using one or more of the compounds disclosed herein and/or one or more compositions disclosed herein to attract psyllids to a device disclosed herein, thereby capturing the psyllids.

Other aspects of the present specification disclose methods of killing psyllids. The disclosed methods may comprises using one or more of the compounds disclosed herein and/or one or more compositions disclosed herein to attract psyllids to a device disclosed herein, thereby killing the psyllids.

Other aspects of the present specification disclose methods of monitoring a psyllid population. The disclosed methods may comprise a) using one or more of the compounds disclosed herein and/or one or more compositions disclosed herein to attract psyllids to a device disclosed herein, thereby capturing the psyllids; b) counting the psyllids to determine a number of captured psyllids; and c) performing a statistical analysis on the number of captured psyllids in order to determine the psyllid population, thereby monitoring the psyllid population.

### DESCRIPTION

Insect chemosensory proteins (CSPs) regulate or control crucial behaviors. The chemosensory system consists of several chemosensory protein (CSP) classes. Chemosensory protein classes that are important in the design of novel insect control products include soluble proteins found in the antennal hemolymph and the maxillary palps, such as odorant binding proteins (OBPs) and sensory appendage proteins (SAPs). OBPs and SAPs are carrier proteins that facilitate the transport of external stimuli such as odor molecules through the aqueous hemolymph of sensory appendages to the surfaces of neuronal cells. There, the protein/odorant molecule complexes bind G-protein coupled receptors (GPCRs) and initiate a signaling cascade that results in a behavioral response to the external odor or stimulus. Insects use chemosensory cues from the environment to control critical behaviors, such as feeding and mating. Thus, insect chemosensory proteins are promising targets for the discovery of novel insect control products based on manipulating insect behavior.

Psyllids use chemosensory cues while foraging that enable these insects to move over considerable distances in order to find citrus and related genera upon which the insects feed, reproduce, and develop. Psyllids of both genders are most strongly attracted to citrus plant odors. In fact, mated psyllids are attracted to citrus odors regardless of the presence or absence of a visual cue. However, despite several years of research, effective psyllid attractants have to date not been identified. The present specification discloses small molecules that are effective psyllid attractants. These psyllid attractants were identified by exploiting the natural chemosensory signaling pathways of psyllids used to attract this insect to citrus. The disclosed psyllid attractants bind to specific chemosensory proteins and affect the behavioral activity on psyllids by acting as “odor mimics” that imitate the chemosensory compounds found in nature. Surprisingly, these small molecule compounds possess improved attractiveness when compared to the natural chemosensory cues produced by citrus plants and provide an ideal opportunity to manipulate psyllid behavior.

Aspects of the present specification disclose, in part, a psyllid attractant. As used herein, a “psyllid attractant” refers to a compound that attracts a psyllid to it source in a manner that mimics the attractant chemosensory cues of a natural compound produced by a citrus plant. A psyllid attractant will typically preferentially bind, without limitation, at least one of the following psyllid chemosensory proteins: odorant-binding protein 1 (OBP1; SEQ ID NO: 1), sensory appendage protein 1 (SAP1; SEQ ID NO: 2), sensory appendage protein 2 (SAP2; SEQ ID NO: 3), sensory appendage protein 3 (SAP3; SEQ ID NO: 4), and sensory appendage protein 4 (SAP4; SEQ ID NO: 5).

Psyllid attractants disclosed herein are useful to attract a psyllid. As used herein, the term “psyllid” refers to a group of insects, commonly called jumping plant lice, belonging to the family Psyllidae which comprises more than 70 genera of insects. Furthermore, as used herein, psyllids refers to any stage of development in the psyllid life-cycle. Non-limiting examples of Psyllidae genera include Acizzia, Agonoscena, Allocaridara, Arytainilla, Blastopsylla, Boreioglycaspis, Cacopsylla, Cryptoneossa, Ctenarytaina, Diaphorina, Eucalyptolyma, Euphyllura, Glycaspis, Heteropsylla, Pachypsylla, Prosopidopsylla, Psylla, Psyllopsis, Retroacizzia, and Tetragonocephela. Non-limiting examples of psyllids for which the psyllid attractants disclosed herein are suitable for the compositions, methods, and uses disclosed herein include Acacia psyllid, Acizzia (Psylla) uncatoides, African citrus psyllid, Trioza erytreae, Apple psyllid, Cacopsylla (Psylla) mali, Apple psyllid, Cacopsylla picta, Apricot psyllid, Cacopsylla pruni, Asian citrus psyllid, Diaphorina citri, Avocado psyllid, Trioza aguacate, Avocado psyllid, Trioza anceps, Avocado psyllid, Trioza godoyae, Avocado psyllid, Trioza perseae, Bay sucker psyllid, Trioza alacris, Blackberry psyllid, Phylloplecta (Trioza) tripunctata, Black-veined psyllid, Cacopsylla melanoneura, Blue gum psyllid, Ctenarytaina eucalypti, Box psyllid, Cacopsylla buxi, Broom psyllid, Arytainilla spartiophila, Carrot psyllid, Bactericera nigricornis, Carrot psyllid, Bactericera trigonica, Carrot psyllid, Trioza apicalis, Cottony ash psyllid, Psyllopsis discrepans, Durian psyllid, Allocaridara malayensis, Eucalyptus shoot psyllid, Blastopsylla occidentalis, False-mastic psyllid, Ceropsylla sideroxyli, Fig psyllid (Moreton Bay fig sucker, Mycopsylla fici, Hackberry bud gall psyllid, Pachypsylla celtidisgemma, Hackberry nipple gall psyllid, Pachypsylla celtidismamma, Hackberry blister gall psyllid, Pachypsylla celtidisvesicula, Hackberry petiole gall psyllid, Pachypsylla venusta, Leucaena psyllid, Heteropsylla cubana, Lemon gum psyllid, Cryptoneossa triangular, Lillypilly psyllid (Eugenia psyllid), Trioza eugeniae, Melaleuca psyllid, Boreioglycaspis melaleucae, Mesquite psyllid, Heteropsylla texana, Mesquite psyllid, Prosopidopsylla flava, Olive psyllid, Euphyllura olivine, Common pear psyllid, Cacopsylla (Psylla) pyricola, Pear psyllid, Cacopsylla (Psylla) pyrisuga, Pear psyllid, Cacopsylla (Psylla) pyri, Peppertree psyllid, Calophya schini, Peppertree psyllid, Calophya rubra, Persimmon psyllid, Trioza diospyri, Common Pistachio psyllid, Agonoscena pistaciae, Pistachio psyllid, Agonoscena targionii, Pittosporum psyllid, Trioza vitreoradiata, Potato/Tomato psyllid, Bactericera (Paratrioza) cockerelli, Red gum lerp psyllid, Glycaspis brimblecombei, Spotted gum lerp psyllid, Eucalyptolyma maideni, Sugarberry psyllid, Tetragonocephela flava, and Trumpet tree psyllid, Trioza tabebuiae.

In one embodiment, a psyllid attractant disclosed herein substantially mimics the attractant chemosensory cues of a natural compound produced by a citrus plant. In an aspect of this embodiment, a psyllid attractant disclosed herein substantially mimics the attractant chemosensory cues of (R)-(+)-Limonene. In another aspect of this embodiment, a psyllid attractant disclosed herein substantially mimics the attractant chemosensory cues of Petitgrain.

A psyllid attractant disclosed herein may be characterized by it binding affinity. Binding affinity can be described by an equilibrium dissociation constant (KD), which is defined as the ratio Kd/Ka at equilibrium; where Ka is the association rate constant of the psyllid attractant and kd is the dissociation rate constant of the psyllid attractant. Binding affinity is determined by both the association and the dissociation and alone neither high association or low dissociation can ensure high affinity. The association rate constant (Ka), or on-rate constant (Kon), measures the number of binding events per unit time, or the propensity of the psyllid attractant and the psyllid OBP or SAP to associate reversibly into its attractant-OBP/SAP complex. The association rate constant is expressed in M⁻¹ s⁻¹. The larger the association rate constant, the more rapidly the psyllid attractant binds to its psyllid OBP or SAP, or the higher the binding affinity between psyllid attractant and psyllid OBP or SAP. The dissociation rate constant (Kd), or off-rate constant (Koff), measures the number of dissociation events per unit time propensity of an attractant-OBP/SAP complex to separate (dissociate) reversibly into its component molecules, namely the psyllid attractant and the psyllid OBP or SAP. The dissociation rate constant is expressed in s⁻¹. The smaller the dissociation rate constant, the more tightly bound the psyllid attractant is to its psyllid OBP or SAP, or the higher the binding affinity between psyllid attractant and psyllid OBP or SAP. The equilibrium dissociation constant (KD) measures the rate at which new attractant-OBP/SAP complexes formed equals the rate at which attractant-OBP/SAP complexes dissociate at equilibrium. The equilibrium dissociation constant is expressed in M, and is defined as Koff/Kon=[L]×[R]/[L+R], where [L] is the molar concentration of the psyllid attractant, [R] is the molar concentration of the psyllid OBP or SAP, and [L+R] is the of molar concentration of the attractant-OBP/SAP complex, where all concentrations are of such components when the system is at equilibrium. The smaller the equilibrium dissociation constant, the more tightly bound the psyllid attractant is to its psyllid OBP or SAP, or the higher the binding affinity between psyllid attractant and psyllid OBP or SAP.

In an embodiment, a psyllid attractant disclosed herein has a binding affinity that is substantially the same as the binding affinity of the natural ligand for that psyllid OBP or SAP. In aspects of this embodiment, a psyllid attractant disclosed herein has a binding affinity for a psyllid OBP or SAP that is, e.g., about 75%, about 80%, about 85%, about 90%, about 95%, about 97%, or about 100% that of the binding affinity of the natural ligand for that psyllid OBP or SAP. In other aspects of this embodiment, a psyllid attractant disclosed herein has a binding affinity for a psyllid OBP or SAP that is, e.g., at least 75%, at least 80%, at least 85%, at least 90%, at least 95%, or at least 97% that of the binding affinity of the natural ligand for that psyllid OBP or SAP. In other aspects of this embodiment, a psyllid attractant disclosed herein has a binding affinity for a psyllid OBP or SAP that is between, e.g., about 75% to about 97%, about 80% to about 97%, about 85% to about 97%, about 90% to about 97%, about 75% to about 100%, about 80% to about 100%, about 85% to about 100%, or about 90% to about 100% that of the binding affinity of the natural ligand for that psyllid OBP or SAP.

In an embodiment, the binding affinity of a psyllid attractant that binds to a psyllid OBP or SAP has a dissociation equilibrium constant that is greater than the dissociation equilibrium constant of the natural ligand for that psyllid OBP or SAP by, e.g., at least one-fold, at least two-fold, at least three-fold, at least four fold, at least five-fold, at least six-fold, at least seven-fold, at least eight-fold, at least nine-fold, at least 10-fold, at least 20-fold, at least 30-fold, at least 40-fold, at least 50-fold, at least 60-fold, at least 70-fold, at least 80-fold, at least 90-fold, at least 100-fold, at least 125-fold, at least 150-fold, at least 175-fold, or at least 200-fold.

In another embodiment, a psyllid attractant disclosed herein has a binding affinity that is substantially the same as the binding affinity of (R)-(+)-Limonene for that psyllid OBP or SAP. In aspects of this embodiment, a psyllid attractant disclosed herein has a binding affinity for a psyllid OBP or SAP that is, e.g., about 75%, about 80%, about 85%, about 90%, about 95%, about 97%, or about 100% that of the binding affinity of (R)-(+)-Limonene for that psyllid OBP or SAP. In other aspects of this embodiment, a psyllid attractant disclosed herein has a binding affinity for a psyllid OBP or SAP that is, e.g., at least 75%, at least 80%, at least 85%, at least 90%, at least 95%, or at least 97% that of the binding affinity of (R)-(+)-Limonene for that psyllid OBP or SAP. In other aspects of this embodiment, a psyllid attractant disclosed herein has a binding affinity for a psyllid OBP or SAP that is between, e.g., about 75% to about 97%, about 80% to about 97%, about 85% to about 97%, about 90% to about 97%, about 75% to about 100%, about 80% to about 100%, about 85% to about 100%, or about 90% to about 100% that of the binding affinity of (R)-(+)-Limonene for that psyllid OBP or SAP.

In an embodiment, the binding affinity of a psyllid attractant that binds to a psyllid OBP or SAP has a dissociation equilibrium constant that is greater than the dissociation equilibrium constant of (R)-(+)-Limonene for that psyllid OBP or SAP by, e.g., at least one-fold, at least two-fold, at least three-fold, at least four fold, at least five-fold, at least six-fold, at least seven-fold, at least eight-fold, at least nine-fold, at least 10-fold, at least 20-fold, at least 30-fold, at least 40-fold, at least 50-fold, at least 60-fold, at least 70-fold, at least 80-fold, at least 90-fold, at least 100-fold, at least 125-fold, at least 150-fold, at least 175-fold, or at least 200-fold.

In another embodiment, a psyllid attractant disclosed herein has a binding affinity that is substantially the same as the binding affinity of Petitgrain for that psyllid OBP or SAP. In aspects of this embodiment, a psyllid attractant disclosed herein has a binding affinity for a psyllid OBP or SAP that is, e.g., about 75%, about 80%, about 85%, about 90%, about 95%, about 97%, or about 100% that of the binding affinity of Petitgrain for that psyllid OBP or SAP. In other aspects of this embodiment, a psyllid attractant disclosed herein has a binding affinity for a psyllid OBP or SAP that is, e.g., at least 75%, at least 80%, at least 85%, at least 90%, at least 95%, or at least 97% that of the binding affinity of Petitgrain for that psyllid OBP or SAP. In other aspects of this embodiment, a psyllid attractant disclosed herein has a binding affinity for a psyllid OBP or SAP that is between, e.g., about 75% to about 97%, about 80% to about 97%, about 85% to about 97%, about 90% to about 97%, about 75% to about 100%, about 80% to about 100%, about 85% to about 100%, or about 90% to about 100% that of the binding affinity of Petitgrain for that psyllid OBP or SAP.

In an embodiment, the binding affinity of a psyllid attractant that binds to a psyllid OBP or SAP has a dissociation equilibrium constant that is greater than the dissociation equilibrium constant of Petitgrain for that psyllid OBP or SAP by, e.g., at least one-fold, at least two-fold, at least three-fold, at least four fold, at least five-fold, at least six-fold, at least seven-fold, at least eight-fold, at least nine-fold, at least 10-fold, at least 20-fold, at least 30-fold, at least 40-fold, at least 50-fold, at least 60-fold, at least 70-fold, at least 80-fold, at least 90-fold, at least 100-fold, at least 125-fold, at least 150-fold, at least 175-fold, or at least 200-fold.

In another embodiment, the binding affinity of a psyllid attractant that binds to a psyllid OBP or SAP can have an association rate constant of, e.g., less than 1×10⁵ M⁻¹ s⁻¹, less than 1×10⁶ M⁻¹ s⁻¹, less than 1×10⁷ M⁻¹ s⁻¹, or less than 1×10⁸ s⁻¹. In another embodiment, the binding affinity of a psyllid attractant that binds to a psyllid OBP or SAP can have an association rate constant of, e.g., more than 1×10⁵ M⁻¹ s⁻¹, more than 1×10⁶ M⁻¹ s⁻¹, more than 1×10⁷ M⁻¹ s⁻¹, or more than 1×10⁸ M⁻¹ s⁻¹. In other aspects, t the binding affinity of a psyllid attractant that binds to a psyllid OBP or SAP can have an association rate constant between 1×10⁵ M⁻¹ s⁻¹ to 1×10⁸ M⁻¹ s⁻¹, 1×10⁶ M⁻¹ s⁻¹ to 1×10⁸ M⁻¹ s⁻¹, 1×10⁵ M⁻¹ s⁻¹ to 1×10⁷ M⁻¹ s⁻¹, or 1×10⁶ M⁻¹ s⁻¹ to 1×10⁷ M⁻¹ s⁻¹.

In another embodiment, the binding affinity of a psyllid attractant that binds to a psyllid OBP or SAP can have a dissociation rate constant of less than 1×10⁻³ s⁻¹, less than 1×10⁻⁴ s⁻¹, or less than 1×10⁻⁵ s⁻¹. In other aspects of this embodiment, the binding affinity of a psyllid attractant that binds to a psyllid OBP or SAP can have a dissociation rate constant of, e.g., less than 1.0×10⁻⁴ s⁻¹, less than 2.0×10⁻⁴ s⁻¹, less than 3.0×10⁻⁴ s⁻¹, less than 4.0×10⁻⁴ s⁻¹, less than 5.0×10⁻⁴ s⁻¹, less than 6.0×10⁻⁴ s⁻¹, less than 7.0×10⁻⁴ s⁻¹, less than 8.0×10⁻⁴ s⁻¹, or less than 9.0×10⁻⁴ s⁻¹. In another embodiment, the binding affinity of a psyllid attractant that binds to a psyllid OBP or SAP can have a dissociation rate constant of, e.g., more than 1×10⁻³ s⁻¹, more than 1×10⁻⁴ s⁻¹, or more than 1×10⁻⁵ s⁻¹. In other aspects of this embodiment, the binding affinity of a psyllid attractant that binds to a psyllid OBP or SAP can have a dissociation rate constant of, e.g., more than 1.0×10⁻⁴ s⁻¹, more than 2.0×10⁻⁴ s⁻¹, more than 3.0×10⁻⁴ s⁻¹, more than 4.0×10⁻⁴ s⁻¹, more than 5.0×10⁻⁴ s⁻¹, more than 6.0×10⁻⁴ s⁻¹, more than 7.0×10⁻⁴ s⁻¹, more than 8.0×10⁻⁴ s⁻¹, or more than 9.0×10⁻⁴ s⁻¹.

In another embodiment, the binding affinity of a psyllid attractant that binds to a psyllid OBP or SAP can have an equilibrium dissociation constant of less than 0.500 nM. In aspects of this embodiment, the binding affinity of a psyllid attractant that binds to a psyllid OBP or SAP can have an equilibrium dissociation constant of, e.g., less than 0.500 nM, less than 0.450 nM, less than 0.400 nM, less than 0.350 nM, less than 0.300 nM, less than 0.250 nM, less than 0.200 nM, less than 0.150 nM, less than 0.100 nM, or less than 0.050 nM. In another embodiment, the binding affinity of a psyllid attractant that binds to a psyllid OBP or SAP can have an equilibrium dissociation constant of more than 0.500 nM. In aspects of this embodiment, the binding affinity of a psyllid attractant that binds to a psyllid OBP or SAP can have an equilibrium dissociation constant of, e.g., more than 0.500 nM, more than 0.450 nM, more than 0.400 nM, more than 0.350 nM, more than 0.300 nM, more than 0.250 nM, more than 0.200 nM, more than 0.150 nM, more than 0.100 nM, or more than 0.050 nM.

In another embodiment, a psyllid attractant is a compound having a structure of formula I:

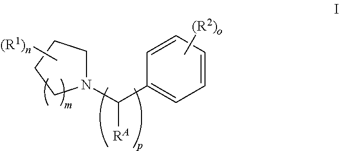

wherein each R^{A} and each R¹ are independently R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃; m is 0, 1, 2, 3 or 4; n is 0, 1, 2, 3, 4, 5 or 6; p is 0, 1, 2, 3, 4, 5 or 6; and o is 0, 1, 2, 3, 4, or 5.

As used herein the term “alkyl” has the broadest meaning generally understood in the art, and may include a moiety composed of carbon and hydrogen containing no double or triple bonds. Alkyl may be linear alkyl, branched alkyl, cycloalkyl, or a combination thereof, and in some embodiments, may contain from one to thirty-five carbon atoms. In aspects of this embodiment, alkyl may include C₁₋₁₀ linear alkyl, such as methyl (—CH₃), ethyl (—CH₂CH₃), n-propyl (—CH₂CH₂CH₃), n-butyl (—CH₂CH₂CH₂CH₃), n-pentyl (—CH₂CH₂CH₂CH₂CH₃), n-hexyl (—CH₂CH₂CH₂CH₂CH₂CH₃), etc.; C₃₋₁₀ branched alkyl, such as C₃H₇ (e.g. iso-propyl), C₄H₉ (e.g. branched butyl isomers), C₅H₁₁ (e.g. branched pentyl isomers), C₆H₁₃ (e.g. branched hexyl isomers), C₇H₁₅ (e.g. heptyl isomers), etc.; C₃₋₁₀ cycloalkyl, such as C₃H₅ (e.g. cyclopropyl), C₄H₇ (e.g. cyclobutyl isomers such as cyclobutyl, methylcyclopropyl, etc.), C₅H₁₁ (e.g. cyclopentyl isomers such as cyclopentyl, methylcyclobutyl, dimethylcyclopropyl, etc.) C₆H₁₁ (e.g. cyclohexyl isomers), C₇H₁₃ (e.g. cycloheptyl isomers), etc.; and the like.

With respect to an optionally substituted moiety such as optionally substituted alkyl, a phrase such as “optionally substituted alkyl” refers to an alkyl that may be unsubstituted, or may have 1 or more substituents, and does not limit the number of carbon atoms in any substituent. A phrase such as “C₁₋₁₂ optionally substituted alkyl” refers to unsubstituted C₁₋₁₂ alkyl, or substituted alkyl wherein both the alkyl parent and all substituents have from 1-12 carbon atoms. Similar conventions may be applied to other optionally substituted moieties such as aryl and heteroaryl.

Substituents on alkyl may be the same as those described generally above, except that alkyl may not have an alkyl substituent. In some embodiments, substituents on alkyl are independently selected from F, Cl, Br, I, OH, NH, ═O, etc.

As used herein, the term “alkoxy” includes —O-alkyl, such as —OCH₃, —OC₂H₅, —OC₃H₇ (e.g. propoxy isomers such as isopropoxy, n-propoxy, etc.), —OC₄H₉ (e.g. butyoxy isomers), —OC₅H₁₁ (e.g. pentoxy isomers), —OC₆H₁₃ (e.g. hexoxy isomers), —OC₇H₁₅ (e.g. heptoxy isomers), etc.

In an aspect of this embodiment, p can be 1 and the compound can have a structure of formula II

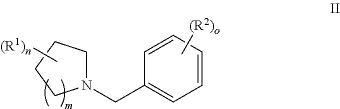

wherein each R¹ is independently R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃; m is 0, 1, 2, 3 or 4; n is 0, 1, 2, 3, 4, 5 or 6; and o is 0, 1, 2, 3, 4, or 5.

In another aspect of this embodiment, m can be 2 and the compound can have a structure of formula III:

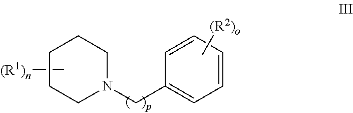

wherein each R¹ is independently R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃; n is 0, 1, 2, 3, 4, 5 or 6; p is 0, 1, 2, 3, 4, 5 or 6; and o is 0, 1, 2, 3, 4, or 5.

In another aspect of this embodiment, m can be 2 and the compound can have a structure of formula IV:

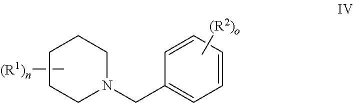

wherein each R¹ is independently R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃; n is 0, 1, 2, 3, 4, 5 or 6; and o is 0, 1, 2, 3, 4, or 5.

In yet another aspect of this embodiment, m can be 3 and the compound can have a structure of formula V:

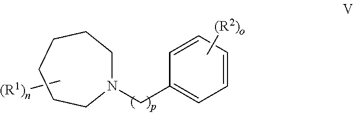

wherein each R¹ is independently R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃; n is 0, 1, 2, 3, 4, 5 or 6; p is 0, 1, 2, 3, 4, 5 or 6; and o is 0, 1, 2, 3, 4, or 5.

In still another aspect of this embodiment, the compound can have a structure of formula VI:

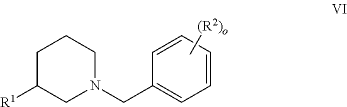

wherein R¹ is R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃; and o is 0, 1, 2, 3, 4, or 5.

In a further aspects of this embodiment, the compound can have a structure of compound 1:

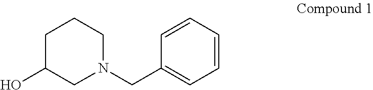 [SMILES: OC1CCCN(Cc2ccccc2)C1]

In a further aspects of this embodiment, the compound can have a structure of formula VII:

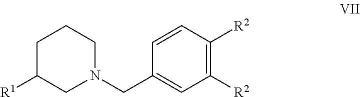

wherein R¹ is R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; and R³ and R⁴ are each independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃.

In some embodiments, a psyllid attractant may be a compound having a structure of formula VIIB:

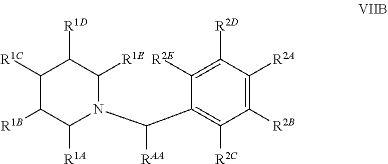

R^{1A} may be R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴.

With respect to R^{1A-E}, R^{AA}, and R^{2A-E}, each R³ and each R⁴ are independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃.

In some embodiments, each R³ and R⁴ of R^{1A} may be H, CH₃, C₂H₅, C₃H₇, cyclopropyl, or CF₃. In some embodiments, R^{1A} is H.

R^{1B} may be R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴. In some embodiments, R³ and R⁴ of R^{1B} may be H, OH, CH₃, C₂H₅, C₃H₇, cyclopropyl, or CF₃. In some embodiments, R^{1B} is H or OH.

R^{1C} may be R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴. In some embodiments, R³ and R⁴ of R^{1C} may be H, CH₃, C₂H₅, C₃H₇, cyclopropyl, or CF₃. In some embodiments, R^{1C} is H.

R^{1D} may be R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴. In some embodiments, R³ and R⁴ of R^{1D} may be H, CH₃, C₂H₅, C₃H₇, cyclopropyl, or CF₃. In some embodiments, R^{1D} is H.

R^{1E} may be R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴. In some embodiments, R³ and R⁴ of R^{1E} may be H, CH₃, C₂H₅, C₃H₇, cyclopropyl, or CF₃. In some embodiments, R^{1E} is H.

R^{AA} may be R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴. In some embodiments, R³ and R⁴ of R^{AA} may be H, CH₃, C₂H₅, C₃H₇, cyclopropyl, or CF₃. In some embodiments, R^{AA} is H.

R^{2A} may be R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴. In some embodiments, R³ and R⁴ of R^{2A} may be H, CH₃, C₂H₅, C₃H₇, cyclopropyl, or CF₃. In some embodiments, R^{2A} is H.

R^{2B} may be R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴. In some embodiments, R³ and R⁴ of R^{2B} may be H, CH₃, C₂H₅, C₃H₇, cyclopropyl, or CF₃. In some embodiments, R^{2B} is H.

R^{2C} may be R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴. In some embodiments, R³ and R⁴ of R^{2C} may be H, CH₃, C₂H₅, C₃H₇, cyclopropyl, or CF₃. In some embodiments, R^{2C} is H.

R^{2D} may be R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴. In some embodiments, R³ and R⁴ of R^{2D} may be H, CH₃, C₂H₅, C₃H₇, cyclopropyl, or CF₃. In some embodiments, R^{2D} is H.

R^{2E} may be R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴. In some embodiments, R³ and R⁴ of R^{2E} may be H, CH₃, C₂H₅, C₃H₇, cyclopropyl, or CF₃. In some embodiments, R^{2E} is H.

In other aspects of this embodiment, a psyllid attractant is a compound having a structure of formula VIII according to Table 1.

TABLE 1
Psyllid Attractants
VIII

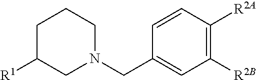

| Compound | R¹ | R^{2A} | R^{2B} |
|---|---|---|---|
| 1 | OH | Cl | Cl |
| 2 | OH | H | Cl |
| 3 | OH | Cl | H |
| 4 | OH | Br | Cl |
| 5 | OH | Cl | Br |
| 6 | OH | Br | Br |
| 7 | OH | Br | H |
| 8 | OH | H | Br |
| 9 | OH | H | H |
| 10 | ═O | Cl | Cl |
| 11 | ═O | H | Cl |
| 12 | ═O | Cl | H |
| 13 | ═O | Br | Cl |
| 14 | ═O | Cl | Br |
| 15 | ═O | Br | Br |
| 16 | ═O | Br | H |
| 17 | ═O | H | Br |
| 18 | ═O | H | H |
| 19 | OH | CH₃ | Cl |
| 20 | OH | Cl | CH₃ |
| 21 | OH | CH₂CH₃ | Cl |
| 22 | OH | Cl | CH₂CH₃ |
| 23 | OH | CH₃ | CH₃ |
| 24 | OH | OH | Cl |
| 25 | OH | Cl | OH |
| 26 | OH | COOCH(CH₃)₂ | Cl |
| 27 | OH | Cl | COOCH(CH₃)₂ |
| 28 | OH | OH | OH |

In yet other aspects of this embodiment, a psyllid attractant is a compound having a structure of formula IX according to Table 2.

TABLE 2
Psyllid Attractants
IX

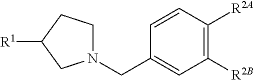

| Compound | R¹ | R^{2A} | R^{2B} |
|---|---|---|---|
| 1 | OH | Cl | Cl |
| 2 | OH | H | Cl |
| 3 | OH | Cl | H |
| 4 | OH | Br | Cl |
| 5 | OH | Cl | Br |
| 6 | OH | Br | Br |
| 7 | OH | Br | H |
| 8 | OH | H | Br |
| 9 | OH | H | H |
| 10 | ═O | Cl | Cl |
| 11 | ═O | H | Cl |
| 12 | ═O | Cl | H |
| 13 | ═O | Br | Cl |
| 14 | ═O | Cl | Br |
| 15 | ═O | Br | Br |
| 16 | ═O | Br | H |
| 17 | ═O | H | Br |
| 18 | ═O | H | H |
| 19 | OH | CH₃ | Cl |
| 20 | OH | Cl | CH₃ |
| 21 | OH | CH₂CH₃ | Cl |
| 22 | OH | Cl | CH₂CH₃ |
| 23 | OH | CH₃ | CH₃ |
| 24 | OH | OH | Cl |
| 25 | OH | Cl | OH |
| 26 | OH | COOCH(CH₃)₂ | Cl |
| 27 | OH | Cl | COOCH(CH₃)₂ |
| 28 | OH | OH | OH |

In another embodiment, a psyllid attractant is a compound having a structure of formula X:

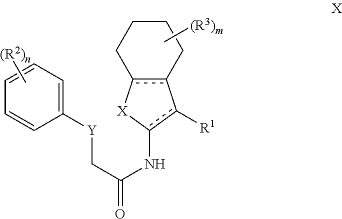

wherein a dashed line represent the presence or absence of a bond; R¹ and R³ are each independently R⁴, optionally substituted aryl, ═O, NR⁴R⁵, OR³, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; each R² is independently R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; R⁴ and R⁵ are each independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃; m is 0, 1, 2, 3, 4, or 5; n is 0, 1, 2, 3, 4, or 5; X is CH₂, O, S, or NH; and Y is CH₂, O, S, or NH.

In an aspect of this embodiment, Y is O and the compound can have a structure of formula XI:

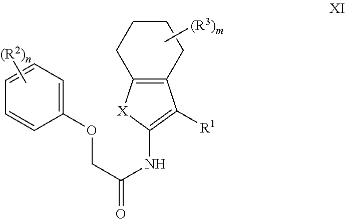

wherein each R³ is independently R⁴, optionally substituted aryl, ═O, NR⁴R⁵, OR³, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; R¹ and each R² is independently R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; R⁴ and R⁵ are each independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃; m is 0, 1, 2, 3, 4, or 5; n is 0, 1, 2, 3, 4, or 5; and X is CH₂, O, S, or NH.

In some embodiments, a psyllid attractant may be a compound having a structure of formula XIB:

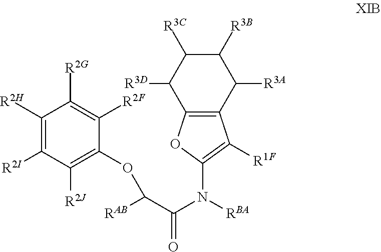

R^{1F} may be R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵.

With respect to R^{1F}, R^{AB}, R^{BA}, R^{2F-J}, and R^{3A-D}, each R⁴ and each R⁵ are independently H, optionally substituted alkyl (such as C₁₋₆ alkyl, including methyl, ethyl, propyl isomers, cyclopropyl, butyl isomers, cyclobutyl isomers, pentyl isomers, cyclopentyl isomers, hexyl isomers, cyclohexyl isomers, etc., or C₁₋₃ alkyl), or CF₃.

In some embodiments, each R⁴ and R⁵ of R^{1F} may be H, CH₃, C₂H₅, C₃H₇, cyclopropyl, or CF₃. In some embodiments, R^{1F} is H or CO₂CH₃.

R^{2F} may be R⁴, optionally substituted aryl, NR⁴R⁵, OR⁴, CN, NO₂, Br, Cl, F, SR⁴SOR⁴, SO₂R⁴, OCOR⁴, CO₂R⁴, COR⁴, CONR⁴R⁵, NR⁴COR⁵, or CSNR⁴R⁵. In some embodiments, each R⁴ and R⁵ of R^{2F} may be H, CH₃, C₂H₅, C₃H₇, cyclopropyl, or CF₃. In some embodiments, R^{2F} is H.

R^{2G} may be R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵. In some embodiments, each R⁴ and R⁵ of R^{2G} may be H, CH₃, C₂H₅, C₃H₇, cyclopropyl, or CF₃. In some embodiments, R^{2G} is H.

R^{2H} may be R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵. In some embodiments, each R⁴ and R⁵ of R^{2H} may be H, CH₃, C₂H₅, C₃H₇, cyclopropyl, or CF₃. In some embodiments, R^{2H} is H.

R^{2I} may be R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵. In some embodiments, each R⁴ and R⁵ of R^{2I} may be H, CH₃, C₂H₅, C₃H₇, cyclopropyl, or CF₃. In some embodiments, R^{2I} is H.

R^{2J} may be R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵. In some embodiments, each R⁴ and R⁵ of R^{2J} may be H, CH₃, C₂H₅, C₃H₇, cyclopropyl, or CF₃. In some embodiments, R^{2J} is H.

R^{3A} may be R⁴, optionally substituted aryl, ═O, NR⁴R⁵, OR^{3A}, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵. In some embodiments, each R⁴ and R⁵ of R^{3A} may be H, CH₃, C₂H₅, C₃H₇, cyclopropyl, or CF₃. In some embodiments, R^{3A} is H.

R^{3B} may be R⁴, optionally substituted aryl, ═O, NR⁴R⁵, OR′, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵. In some embodiments, each R⁴ and R⁵ of R^{3B} may be H, CH₃, C₂H₅, C₃H₇, cyclopropyl, or CF₃. In some embodiments, R^{3B} is H.

R^{3C} may be R⁴, optionally substituted aryl, ═O, NR⁴R⁵, OR^{3C}, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵. In some embodiments, each R⁴ and R⁵ of R^{3C} may be H, CH₃, C₂H₅, C₃H₇, cyclopropyl, or CF₃. In some embodiments, R^{3C} is H.

R^{3D} may be R⁴, optionally substituted aryl, ═O, NR⁴R⁵, OR^{3D}, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵. In some embodiments, each R⁴ and R⁵ of R^{3D} may be H, CH₃, C₂H₅, C₃H₇, cyclopropyl, or CF₃. In some embodiments, R^{3D} is H.

In other aspects of this embodiment, a psyllid attractant is a compound having a structure of formula XII according to Table 3.

TABLE 3
Psyllid Attractants
XII

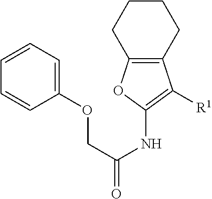

| Compound | R¹ |
|---|---|
| 1 | H |
| 2 | OH |
| 3 | CH₃ |
| 4 | (CH₂)₂CH₃ |
| 5 | Cl |
| 6 | Br |
| 7 | I |
| 8 | CH₃OH |
| 9 | COOCH₃ |
| 10 | COOCH₂CH₃ |
| 11 | COOCH(CH₃)₂ |
| 12 | NH₂ |
| 13 | CN |
| 14 | OCH₃ |
| 15 | OCH₂CH₃ |
| 16 | CH₂Cl |
| 17 | CH₂Br |
| 18 | COOH |
| 19 | NHCH₃ |
| 20 | CONHCH₃ |
| 21 | COH |

In yet another aspect of this embodiment, the compound can have a structure of compound 2:

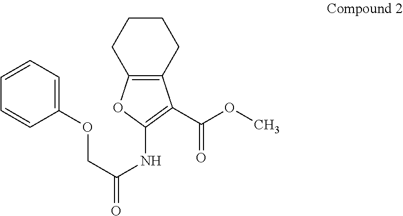 [SMILES: COC(=O)c1c(NC(=O)COc2ccccc2)oc2c1CCCC2]

In another embodiment, a psyllid attractant is one of the following compounds:

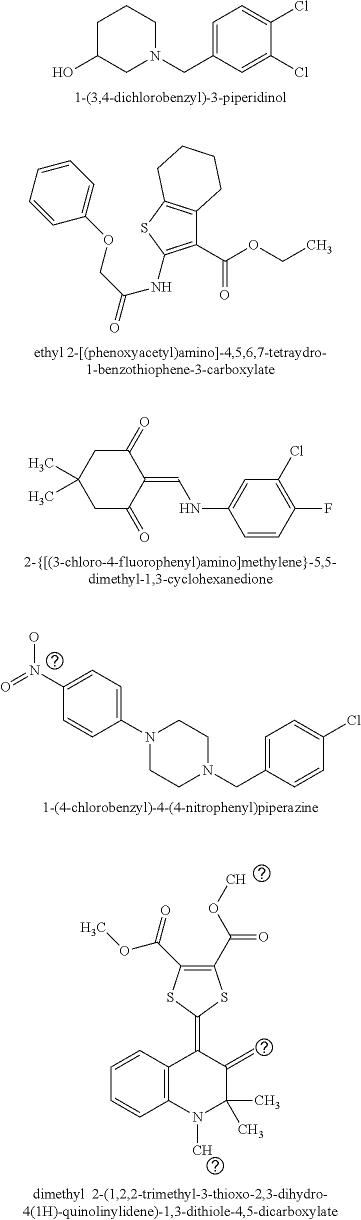

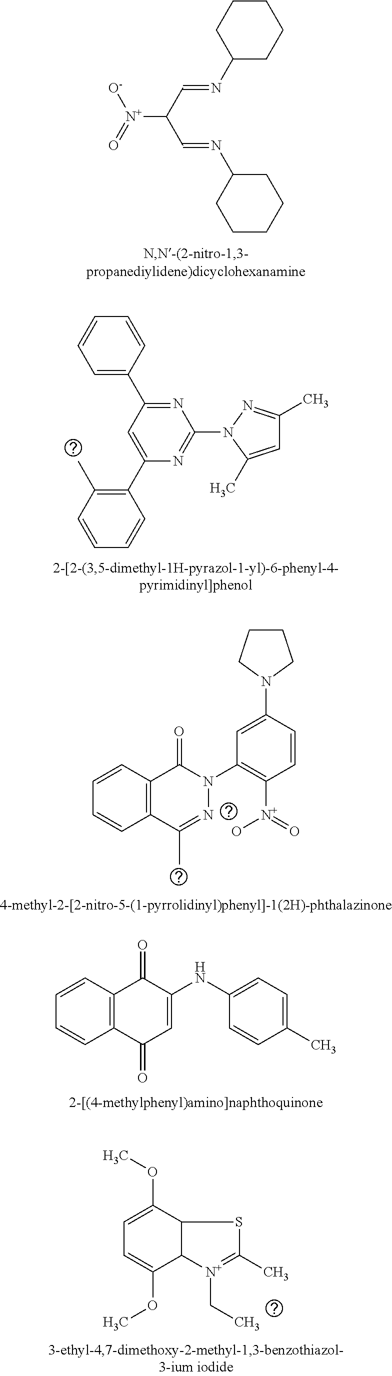

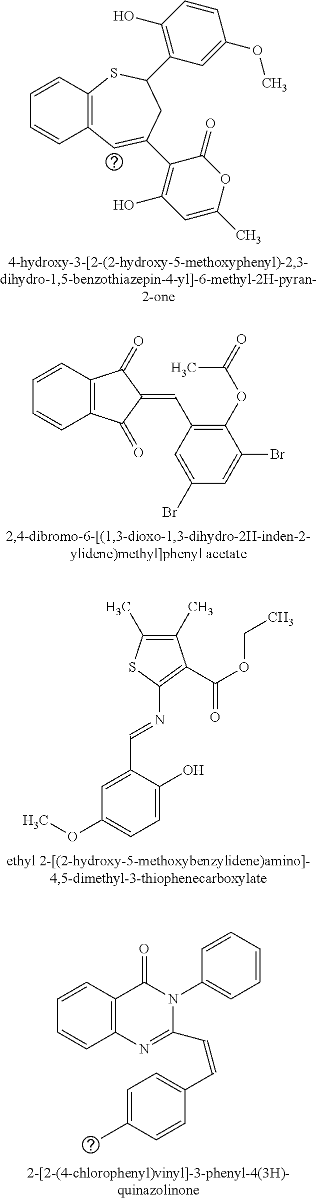

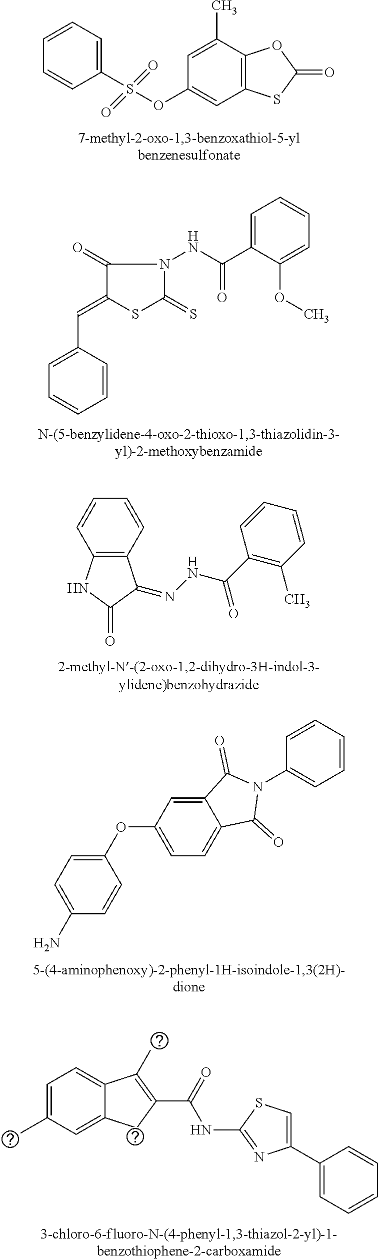

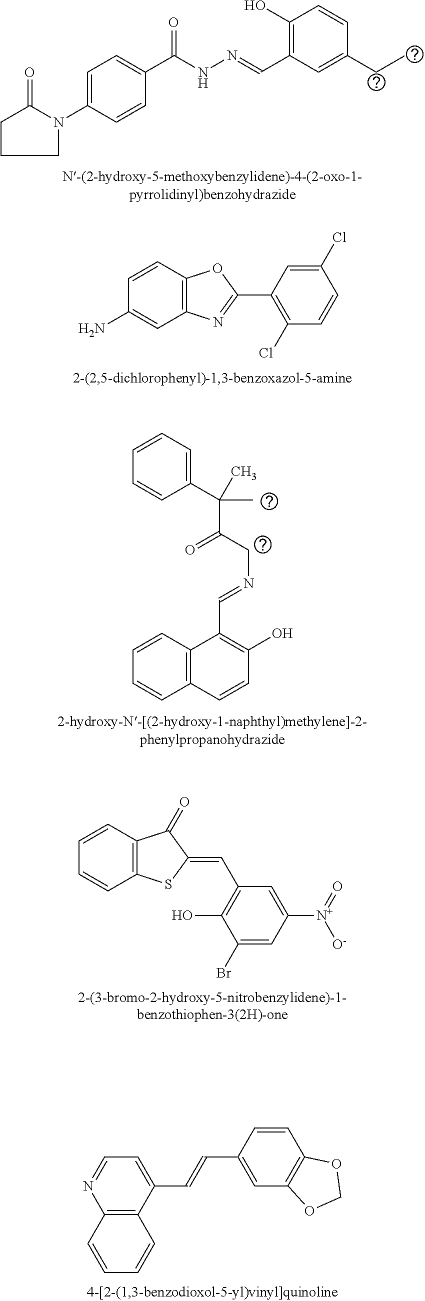 [SMILES: CCc1ccc(O)c(/C=N/NC(=O)c2ccc(N3CCCC3=O)cc2)c1 ; Nc1ccc2oc(-c3cc(Cl)ccc3Cl)nc2c1 ; CC(C)(C(=O)C/N=C/c1c(O)ccc2ccccc12)c1ccccc1 ; O=C1/C(=C/c2cc([N+](=O)[O-])cc(Br)c2O)Sc2ccccc21 ; C(=C\c1ccnc2ccccc12)/c1ccc2c(c1)OCO2]

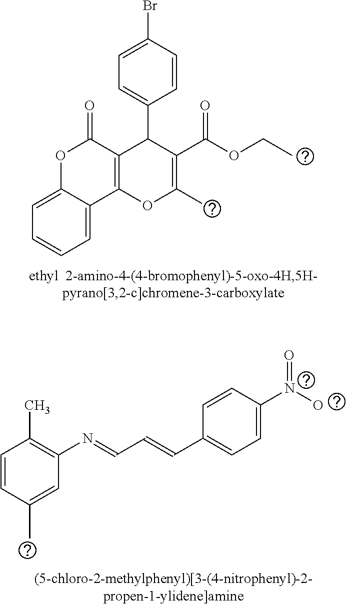

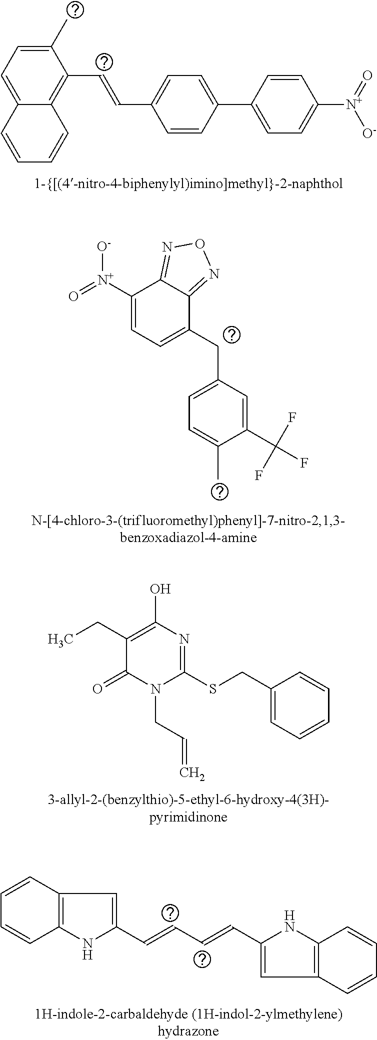 [SMILES: Cc1ccc2ccccc2c1/C=C/c1ccc(-c2ccc([N+](=O)[O-])cc2)cc1 ; Cc1ccc(Cc2ccc([N+](=O)[O-])c3nonc23)cc1C(F)(F)F ; C=CCn1c(SCc2ccccc2)nc(O)c(CC)c1=O ; C(/C=C/c1cc2ccccc2[nH]1)=C\c1cc2ccccc2[nH]1]

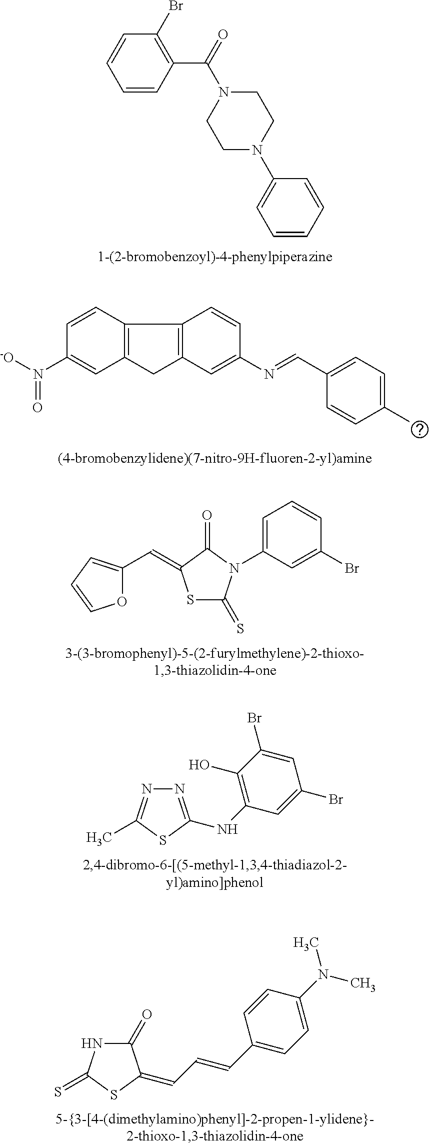

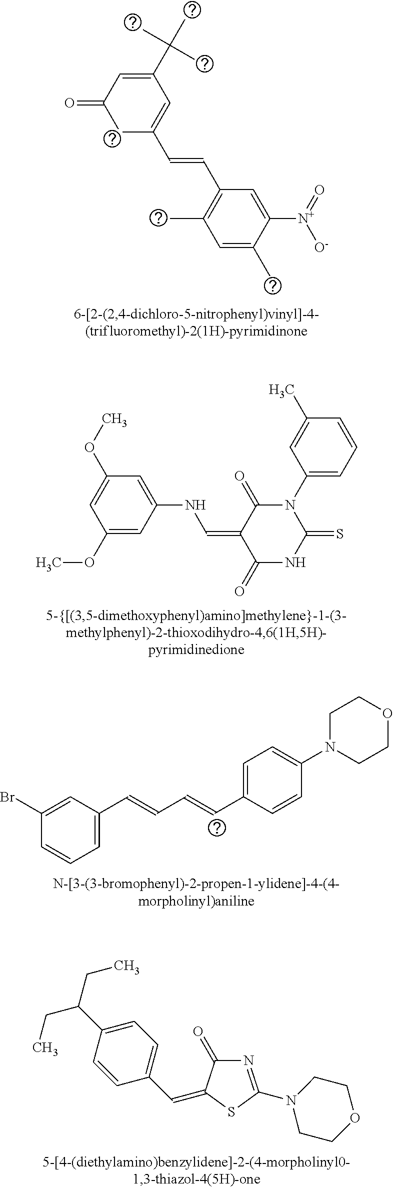 [SMILES: Cc1cc(C)c([N+](=O)[O-])cc1/C=C/C1=CC(C(C)(C)C)=CC(=O)C1 ; COc1cc(N/C=C2/C(=O)NC(=S)N(c3cccc(C)c3)C2=O)cc(OC)c1 ; Brc1cccc(/C=C/C=C/c2ccc(N3CCOCC3)cc2)c1 ; CCC(CC)c1ccc(/C=C2/SC(N3CCOCC3)=NC2=O)cc1]

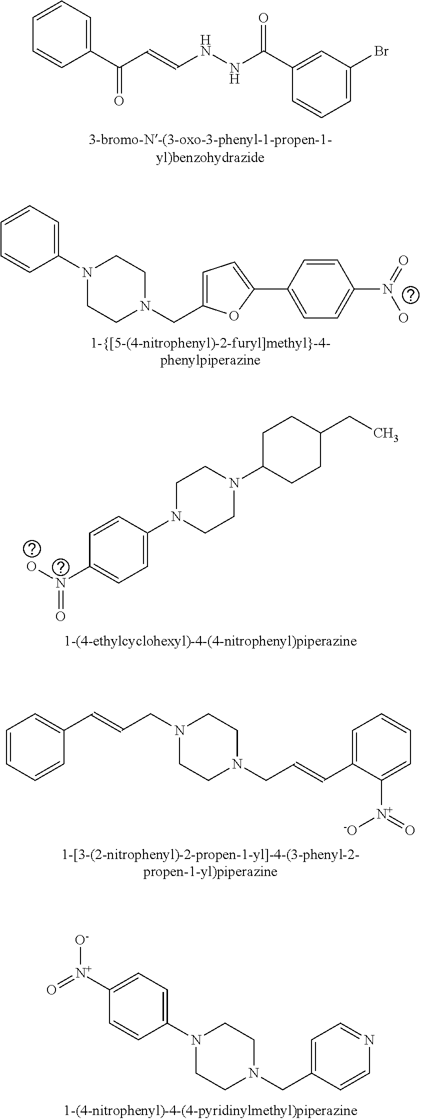

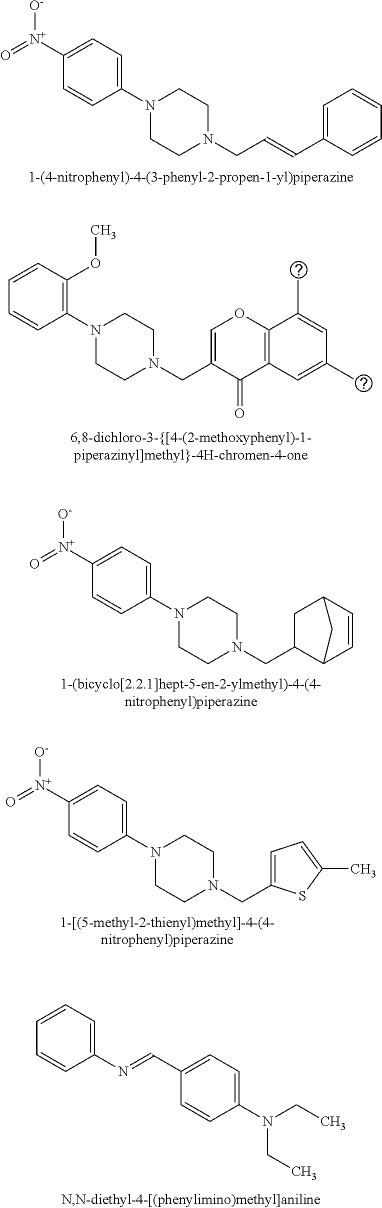 [SMILES: O=[N+]([O-])c1ccc(N2CCN(C/C=C/c3ccccc3)CC2)cc1 ; COc1ccccc1N1CCN(Cc2coc3c(C)cc(C)cc3c2=O)CC1 ; O=[N+]([O-])c1ccc(N2CCN(CC3CC4C=CC3C4)CC2)cc1 ; Cc1ccc(CN2CCN(c3ccc([N+](=O)[O-])cc3)CC2)s1 ; CCN(CC)c1ccc(/C=N/c2ccccc2)cc1]

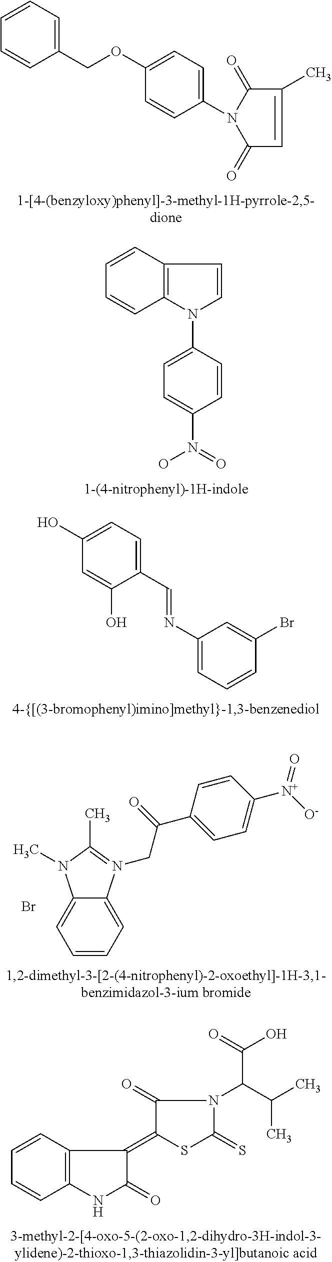

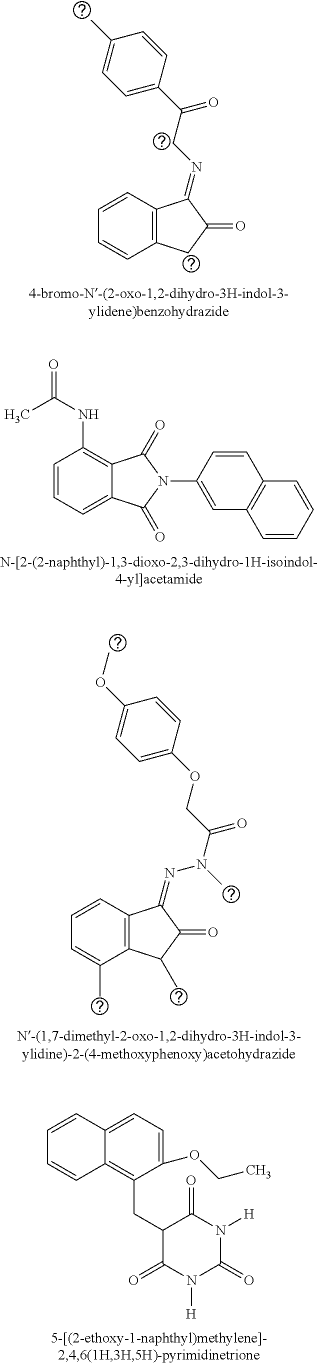 [SMILES: Cc1ccc(C(=O)C/N=C2/C(=O)Cc3ccccc32)cc1 ; CC(=O)Nc1cccc2c1C(=O)N(c1ccc3ccccc3c1)C2=O ; COc1ccc(OCC(=O)N(C)/N=C2\C(=O)C(C)c3c(C)cccc32)cc1 ; CCOc1ccc2ccccc2c1CC1C(=O)NC(=O)NC1=O]

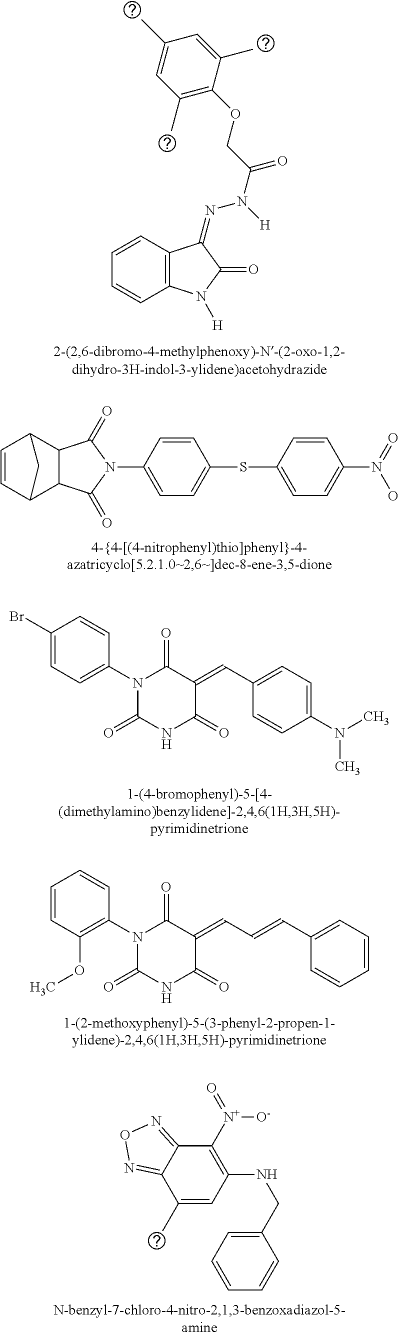

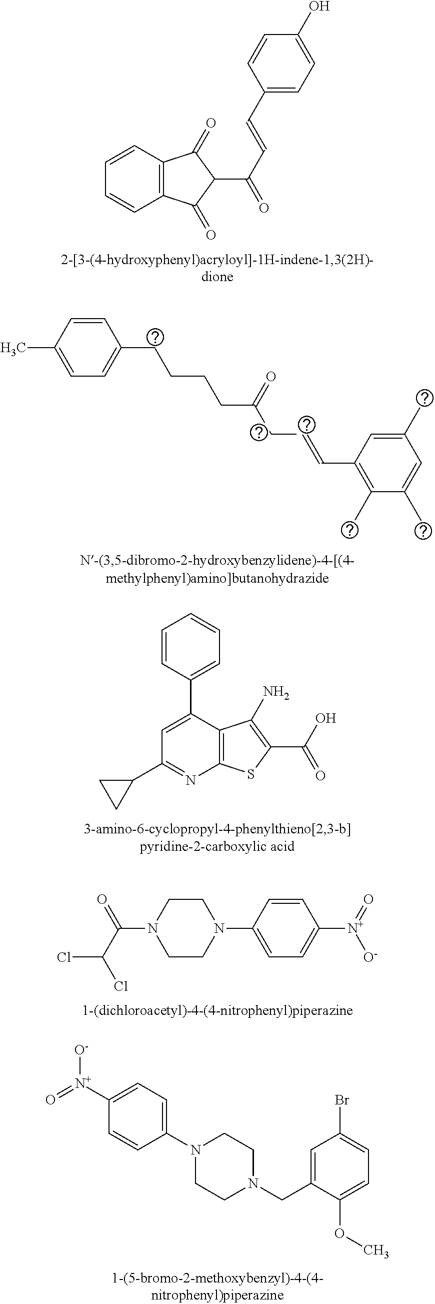

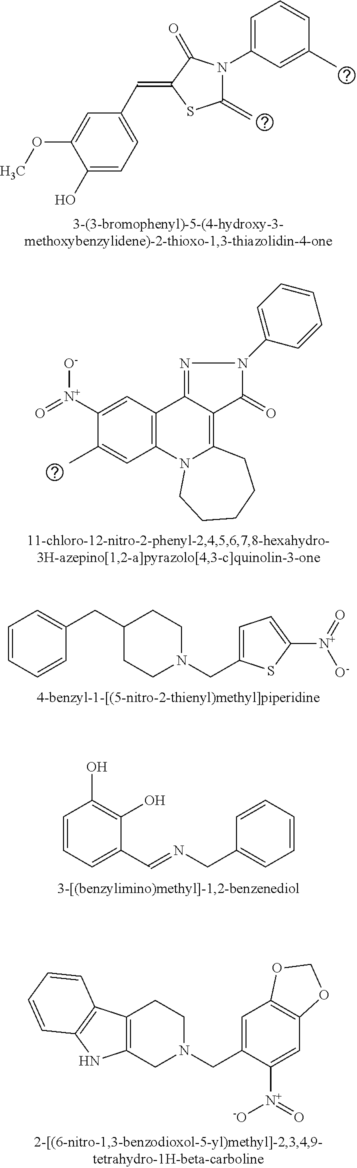

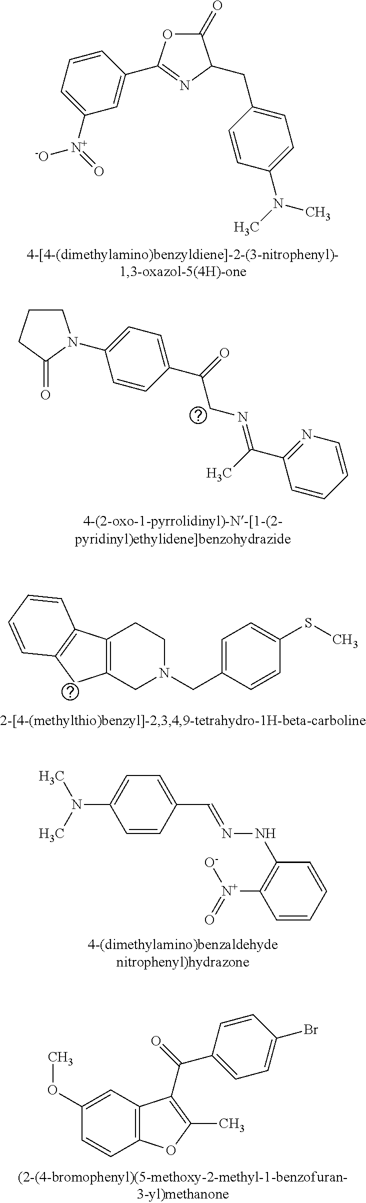

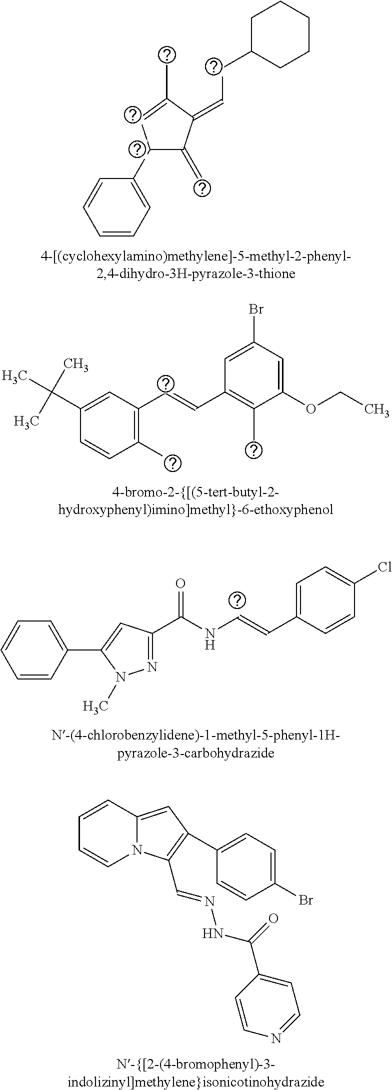

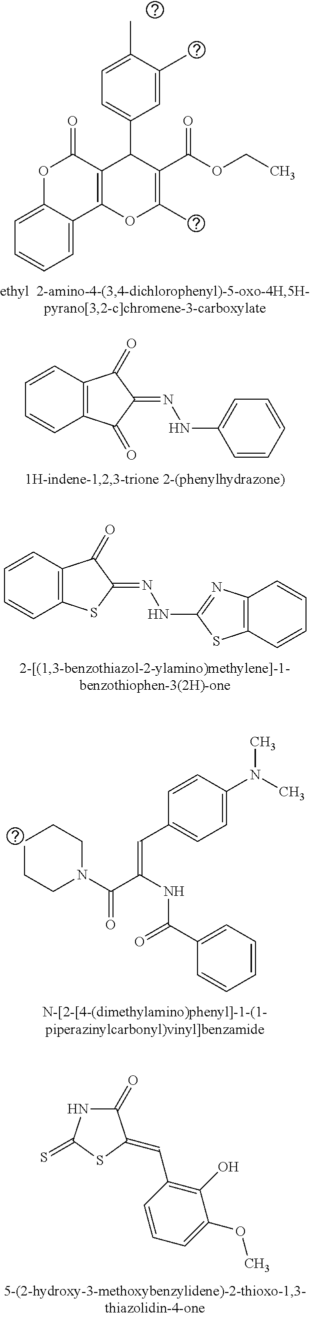 [SMILES: CCOC(=O)C1=C(C)Oc2c(c(=O)oc3ccccc23)C1c1ccc(C)c(C)c1 ; O=c1c(=NNc2ccccc2)c(=O)c2ccccc12 ; O=C1/C(=N/Nc2nc3ccccc3s2)Sc2ccccc21 ; CN(C)c1ccc(/C=C(\NC(=O)c2ccccc2)C(=O)N2CCCCC2)cc1 ; COc1cccc(/C=C2\SC(=S)NC2=O)c1O]

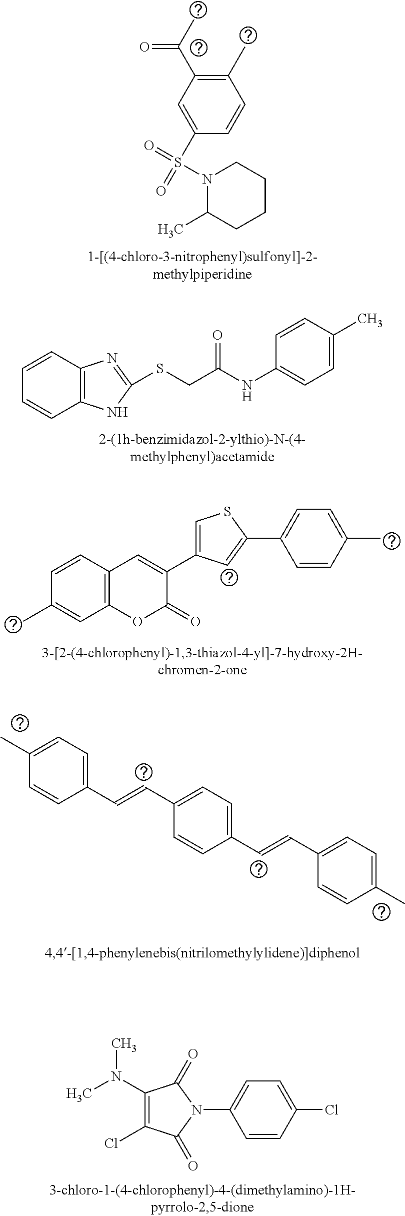

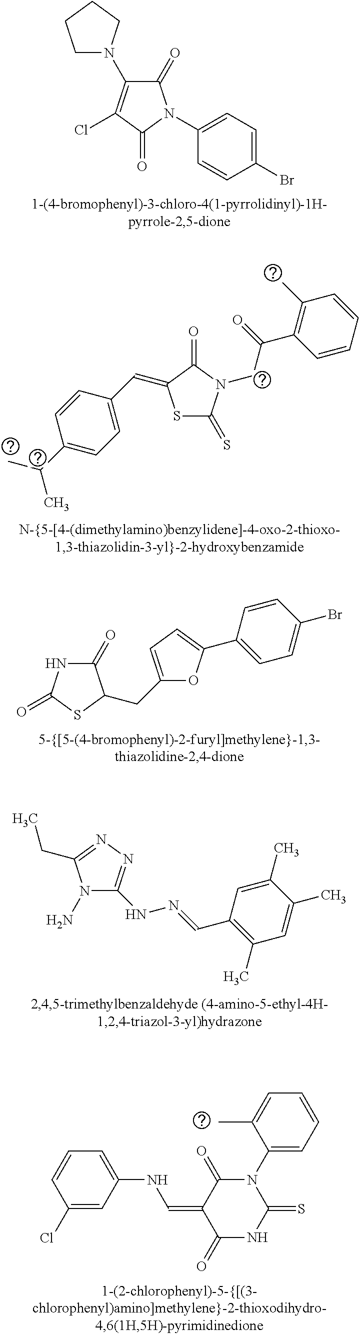 [SMILES: O=C1C(Cl)=C(N2CCCC2)C(=O)N1c1ccc(Br)cc1 ; Cc1ccccc1C(=O)CN1C(=O)/C(=C/c2ccc(C(C)C)cc2)SC1=S ; O=C1NC(=O)C(Cc2ccc(-c3ccc(Br)cc3)o2)S1 ; CCc1nnc(N/N=C/c2cc(C)c(C)cc2C)n1N ; Cc1ccccc1N1C(=O)/C(=C\Nc2cccc(Cl)c2)C(=O)NC1=S]

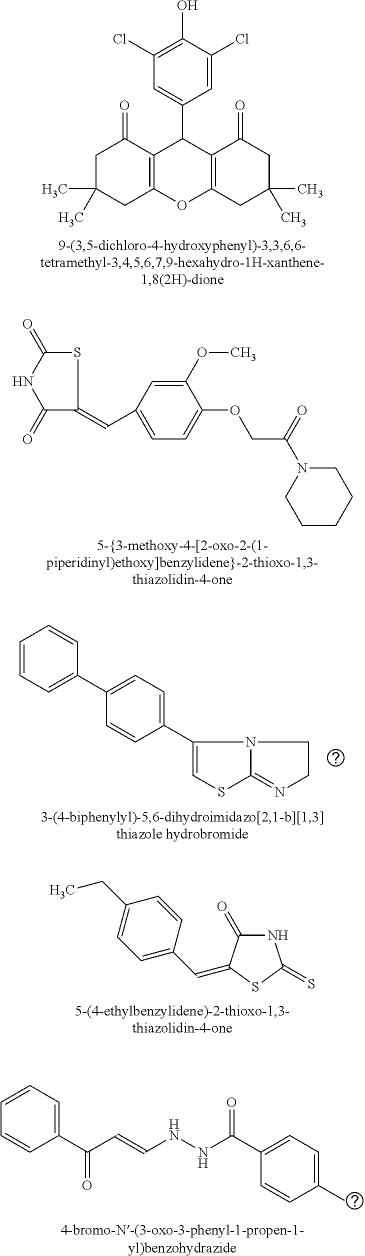

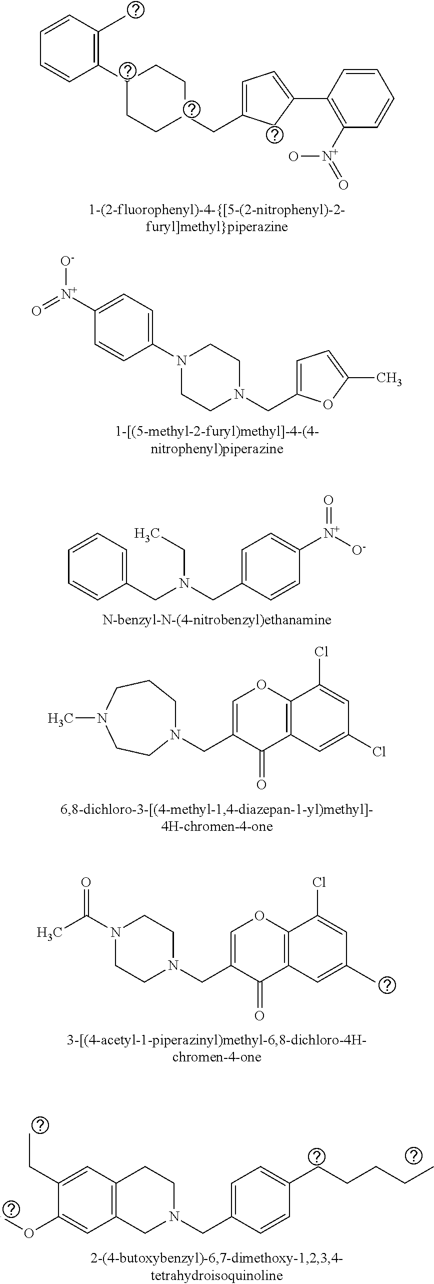

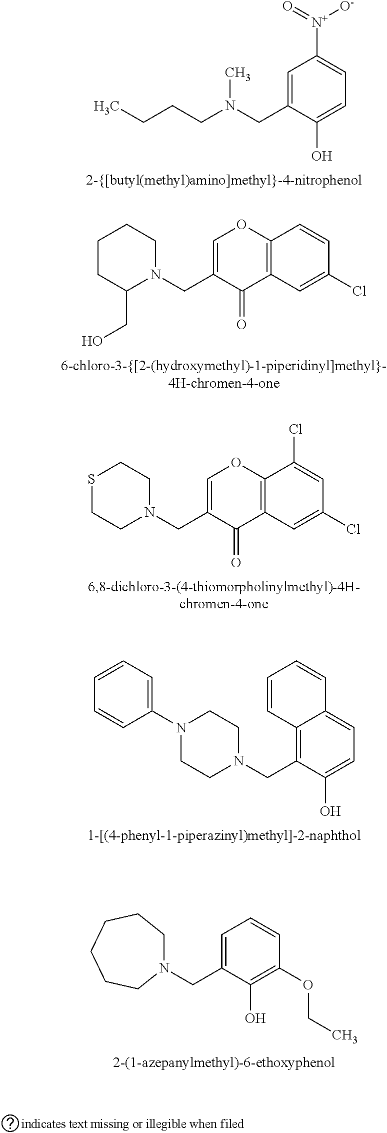 [SMILES: CCCCN(C)Cc1cc([N+](=O)[O-])ccc1O ; O=c1c(CN2CCCCC2CO)coc2ccc(Cl)cc12 ; O=c1c(CN2CCSCC2)coc2c(Cl)cc(Cl)cc12 ; Oc1ccc2ccccc2c1CN1CCN(c2ccccc2)CC1 ; CCOc1cccc(CN2CCCCCC2)c1O]

Aspects of the present specification provide, in part, a composition comprising a psyllid attractant disclosed herein. A composition disclosed herein may comprise a single psyllid attractant disclosed herein or a plurality of psyllid attractant combinations. For instance, a composition may comprise, e.g., two or more different psyllid attractants disclosed herein, three or more different psyllid attractants disclosed herein, four or more different psyllid attractants disclosed herein, five or more different psyllid attractants disclosed herein, or six or more different psyllid attractants disclosed herein.

A composition disclosed herein may optionally comprise additional components such as, e.g., an adhesive, a solvent, a wetting agent, an emulsifying agent, a carrier, a diluent, or a dispersing agent. Such additional components are known to a person of skill in the art. A composition disclosed herein can take any of a variety of dosage forms including, without limitation, a liquid composition, such as, e.g., a solution, suspension, emulsion; a semi-solid composition, such as, e.g., a ointment, cream, balm, foam, gel, salve or a solid composition, such as, e.g., lyophilizate, powder, granule, pellet, capsule; or any other dosage form suitable for applying a psyllid attractant disclosed herein to a location to be treated. In one aspect of this embodiment, in liquid, semi-solid, and solid forms, an amount of a psyllid attractant disclosed herein typically is between about 0.0001% to about 50% about 0.001% to about 10.0% or about 0.01% to about 1.0% by weight of a composition. As used herein, by weight of a composition may be (v/v), (w/w), (w/v), or (v/w). In another aspect of this embodiment, in semi-solid and solid forms, an amount of a psyllid attractant disclosed herein is typically is between about 0.001 mg/L to about 500 mg/L, about 0.01 mg/L to about 100 mg/L, or about 0.1 mg/L to about 50 mg/L.

The amount of a psyllid attractant disclosed herein used in the compositions disclosed herein is an effective amount. As used herein, the term “effective amount” refers to an amount of a psyllid attractant or composition disclosed herein sufficient to attract or direct movement of psyllids to the source of attractant release.

In aspects of this embodiment, a composition comprises a psyllid attractant in an effective amount of, e.g., at least 0.0001%, at least 0.00025%, at least 0.0005%, at least 0.00075%, at least 0.001%, at least 0.0025%, at least 0.005%, at least 0.0075%, at least 0.01%, at least 0.025%, at least 0.05%, at least 0.075%, at least 0.1%, at least 0.25%, at least 0.5%, at least 0.75%, at least 1%, at least 2.5%, at least 5%, at least 7.5%, at least 10%, at least 25%, or at least 50%, by weight of a composition.

In other aspects of this embodiment, a composition comprises a psyllid attractant in an effective amount of between, e.g., about 0.0001% to about 0.001%, about 0.0001% to about 0.01%, about 0.0001% to about 0.1%, about 0.00025% to about 0.0025%, about 0.00025% to about 0.025%, about 0.00025% to about 0.25%, about 0.0005% to about 0.005%, about 0.0005% to about 0.05%, about 0.0005% to about 0.75%, about 0.00075% to about 0.0075%, about 0.00075% to about 0.075%, about 0.00075% to about 0.75%, about 0.001% to about 0.01%, about 0.001% to about 0.1%, about 0.001% to about 1%, about 0.0025% to about 0.025%, about 0.0025% to about 0.25%, about 0.0025% to about 2.5%, about 0.005% to about 0.05%, about 0.005% to about 0.5%, about 0.005% to about 5%, about 0.0075% to about 0.075%, about 0.0075% to about 0.75%, about 0.0075% to about 7.5%, about 0.01% to about 0.1%, about 0.01% to about 1%, about 0.01% to about 10%, about 0.025% to about 0.25%, about 0.025% to about 2.5%, about 0.025% to about 25%, about 0.05% to about 0.5%, about 0.05% to about 5%, about 0.05% to about 50%, about 0.075% to about 0.75%, about 0.075% to about 7.5%, or about 0.075% to about 75%, by weight of a composition. In yet other aspects of this embodiment, a composition comprises a psyllid attractant in an effective amount of between, e.g., about 0.005% to about 0.015%, about 0.0025% to about 0.025%, or about 0.006% to about 0.016%, by weight of a composition.

A composition disclosed herein may optionally comprise an insecticide. Insecticides include oils, emulsifers, detergents, soaps, microorganisms like fungi, bacteria, bacteriophages, and viruses, abrasives, toxins, and poisons. Non-limiting examples of an insecticide include a organochlorine, such as, e.g., Aldrin, Chlordane, Chlordecone, DDT, Dieldrin, Endosulfan, Endrin, Heptachlor, Hexachlorobenzene, Lindane (gamma-hexachlorocyclohexane), Methoxychlor, Mirex, Pentachlorophenol, and TDE; an organophosphate, such as, e.g., Acephate, Azinphos-methyl, Bensulide, Chlorethoxyfos, Chlorpyrifos, Chlorpyriphos-methyl, Diazinon, Dichlorvos (DDVP), Dicrotophos, Dimethoate, Disulfoton, Ethoprop, Fenamiphos, Fenitrothion, Fenthion, Fosthiazate, Malathion, Methamidophos, Methidathion, Mevinphos, Monocrotophos, Naled, Omethoate, Oxydemeton-methyl, Parathion, Parathion-methyl, Phorate, Phosalone, Phosmet, Phostebupirim, Phoxim, Pirimiphos-methyl, Profenofos, Terbufos, Tetrachlorvinphos, Tribufos, and Trichlorfon; a carbamate, such as, e.g., Aldicarb, Bendiocarb, Carbofuran, Carbaryl, Dioxacarb, Fenobucarb, Fenoxycarb, Isoprocarb, Methomyl, and 2-(1-Methylpropyl)phenyl methylcarbamate; a pyrethroid, such as, e.g., Allethrin, Bifenthrin, Cyhalothrin, A-Cyhalothrin, Cypermethrin, Cyfluthrin, Deltamethrin, Etofenprox, Fenvalerate, Permethrin, Phenothrin, Prallethrin, Resmethrin, Tetramethrin, Tralomethrin, and Transfluthrin; a neonicotinoid, such as, e.g., Acetamiprid, Clothianidin, Imidacloprid, Nitenpyram, Nithiazine, Thiacloprid, and Thiamethoxam; and a spinosad, such as, e.g., spinosyn A and spinosyn B.

Aspects of the present specification disclose, in part, a device. As used herein, the term “device” refers to any device designed to attract and/or capture psyllids. Generally, an effective amount of a psyllid attractant or composition disclosed herein is applied to a device disclosed herein so that the psyllids are lured towards or into/onto the device. A device disclosed herein may be a container, holder or other solid support onto or into which a psyllid attractant or composition disclosed herein. A device disclosed herein may be made form any biological or synthetic material, including, without limitation, paper, filter paper, wood, cork, cotton, plastic, polymer, metal, or glass. A device disclosed herein, includes, without limitation, a known insect trap, a lure, a bait and kill trap, a sticky trap, a blunder trap, a volatile-dispenser, a trap-crop, or a trap plant. A device disclosed herein may capture psyllids in a manner designed to keep the captured insects alive or it may capture psyllids in a manner designed to kill the captured insects. A device disclosed herein may lure psyllids in a manner designed to attract psyllids to a location where the psyllids are used as a biocontrol for preventing the infestation of a plant species.

Exemplary devices useful with a psyllid attractant or composition disclosed herein and the methods and uses disclosed herein are describe in, e.g., U.S. Pat. No. 5,713,153, U.S. Pat. No. 6,430,868, U.S. Pat. No. 6,516,558, U.S. Pat. No. 6,637,149, U.S. Pat. No. 6,772,556, U.S. Pat. No. 6,789,352, U.S. Pat. No. 6,792,713, U.S. Pat. No. 6,860,062, U.S. Pat. No. 6,886,292, D561,865, D564,061, D565,145, U.S. Pat. No. 7,481,022, US 2012/0079759, US 2012/0066958, US 2012/0066952, US 2012/0005947, US 2011/0283597, US 2011/0209384, US 2011/0146133, US 2011/0099885, US 2011/0078941, US 2011/0072710, US 2011/0056118, US 2011/0041384, US 2011/0030266, US 2011/0005123, US 2010/0287816, US 2010/0263260, US 2010/0229459, US 2010/0212211, US 2010/0071254, US 2009/0293342, US 2009/0288333, US 2009/0183419, US 20090151228, US 2009/0094883, US 2008/0256843, US 2008/0086932, US 2007/0251139, US 2005/0172542, and US 2005/0144831, each of which is hereby incorporated by reference in its entirety.

In one aspect of this embodiment, a device comprises a surface including an effective amount of a psyllid attractant disclosed herein and an adhesive used to hinder or prevent psyllids from leaving the surface once contact with the surface is made. In another aspect of this embodiment, a device comprises a surface including an effective amount of a psyllid attractant disclosed herein, an adhesive used to hinder or prevent psyllids from leaving the surface once contact with the surface is made, and an insecticide used to kill the captured psyllids. The insecticide may be present in the same composition as the psyllid attractant, present in the same composition as the adhesive, or present as a separate composition in the device. One exemplary example of such a device is a blunder or sticky trap modified to include the psyllid attractant alone or with an insecticide.

In another aspect of this embodiment, a device comprises a container including a chamber operably-connected to an entrance and an effective amount of a psyllid attractant disclosed herein. In another aspect of this embodiment, a device comprises a chamber operably-connected to an entrance, an effective amount of a psyllid attractant disclosed herein, and an insecticide used to kill psyllids that have entered into the chamber. Psyllids may enter into the chamber through the entrance. The entrance may be a simple opening allowing psyllids to freely ingress or egress from the chamber, or the entrance may be designed in a manner that hinders or prevents psyllids to leave the chamber once entered. The insecticide may be present in the same composition as the psyllid attractant, present as a separate composition in the same chamber as the psyllid attractant, or present as a separate composition in a separate part or location of the device. One exemplary example of such a device is a hotel trap modified to include the psyllid attractant alone or with an insecticide.

In another embodiment, a psyllid attractant disclosed herein can be impregnated into a compatible matrix. As used herein, the term “compatible matrix” refers to any material in which one or more psyllid attractants disclosed herein are either soluble or miscible and where the material does not significantly alter or degrade the attractant activity of the one or more psyllid attractant. In aspects of this embodiment, a compatible matrix does not significantly alter or degrade an attractant activity of one or more psyllid attractants over a period of, e.g., at least 7 days, at least 14 days, at least 21 days, at least 28 days, at least 35 days, at least 42 days, at least 49 days, at least 56 days, or at least 63 days. Impregnation of a psyllid attractant into the compatible matrix can be achieved by any well known methods known in the art. For example, a psyllid attractant can be dissolved into a compatible volatile solvent and the resulting solution added to the matrix whereupon evaporation of the solvent results in impregnation of the psyllid attractant into the compatible matrix. In this regard, the matrix can be cotton twine, polymers such as, e.g., polyvinyls, polyisoprenes, polyethylene, polypropylene or copolymers thereof, or polybutenes. In another example, a compatible matrix is thinned by heating and then a psyllid attractant is added directly thereto. The mixture can then be combined with twine or other compatible matrices. A compatible matrix disclosed herein may employed or incorporated into a trap disclosed herein

In another embodiment, a psyllid attractant disclosed herein can be incorporated into a controlled-release device which dispenses the psyllid attractant over time in a regulated or predictable manner. A controlled-release device disclosed herein may be employed or incorporated into a trap disclosed herein.

One type of controlled-release device is a “reservoir” device where the psyllid attractant forms a core surrounded by an inert diffusion barrier. An inert diffusion barrier includes membranes which are non-porous, homogeneous polymeric films, through which transport occurs by a process of dissolution of the permeating species in the polymer at one interface and diffusion down a gradient in thermodynamic activity. These membranes are usually referred to as solution-diffusion membranes. Another class inert diffusion barrier includes the porous and/or fibrous barriers such as, for example, hollow fibers, porous and/or fibrous materials, in which a psyllid attractant diffuses mainly by capillary forces. Other less common reservoir devices are designed to enable diffusion to take place by mechanical pumping or under external forces, such as, e.g., gravity, electrical field, vacuum, or centrifugal forces. A reservoir device can exist in a variety of shapes, and can be degradable or non-degradable.

In an aspect of this embodiment, a reservoir device is a microcapsule comprising a core of a psyllid attractant disclosed herein surrounded by a coating or shell of, e.g., a polyvinyl chloride (PVC)-polyvinyl acetate (PVA) plastic. Size typically varies from about 1 μm to about 1000 μm and can have irregular or geometric shapes. Core payload usually varies from 0.1 to 98 weight percent. Encapsulation processes are often loosely classified as either chemical or mechanical. Examples of chemical processes include but are not limited to complex coacervation, polymer-polymer incompatibility, interfacial polymerization in liquid media, in situ polymerization, in-liquid drying, thermal and ionic gelation in liquid media, desolvation in liquid media, starch-based chemistry processes, trapping in cyclodextrins, and formation of liposomes. Examples of mechanical processes include but are not limited to spray drying, spray chilling, fluidized bed, electrostatic deposition, centrifugal extrusion, spinning disk or rotational suspension separation, annular-jet encapsulation, polymerization at liquid-gas or solid-gas interface, solvent evaporation, pressure extrusion or spraying into solvent extraction bath.

Another type of controlled-release device is a “monolithic” device where a psyllid attractant is dissolved or dispersed throughout a substantially inert matrix from which the psyllid attractant is gradually released. Non-limiting examples of matrices included in a monolithic device include various gels, waxes, gelatins, natural resins, rubbers, elastomers, synthetic and natural polymers. A monolithic device can exist in a variety of shapes, and can be degradable or non-degradable. Size can vary depending on the application. For example, a monolithic device can be produced as a microcapsule having a size of about 1 μm to about 1000 μm with irregular or geometric shapes. As another example, a monolithic device can have a size of about 1 mm to about 10 cm with irregular or geometric shape.

A controlled-release device disclosed herein can be a liquid composition or a solid composition. A liquid sustained-release formulation includes a psyllid attractant disclosed herein, a solvent, and typically further comprise surface active agents to render the composition readily dispersible in water, such agents include a wetting agent, an emulsifying agent, or a dispersing agent. In one embodiment, a liquid form of a sustained-release formulation is an emulsion formulation, such as, e.g., a water in oil (w/o) emulsion or oil in water (o/w) emulsion. Non-limiting examples of oils include vegetable oils and mineral oils. Droplet size can vary from the nanometer scale (colloidal dispersion) to several hundred microns. A variety of surfactants and thickeners are usually incorporated in the formulation to modify the size of the droplets, stabilize the emulsion, and modify the release.

A solid form of controlled-release device comprises a solid substrate like porous particulates such as silica, perlite, talc, clay, pyrophyllite, diatomaceous earth, gelatin and gels, polymers (e.g., polyurea, polyurethane, polyamide, polyester, etc.), polymeric particles, or cellulose. These include, for example, hollow fibers, hollow tubes or tubing which release a psyllid attractant disclosed herein through the walls, capillary tubing which releases the compound out of an opening in the tubing, polymeric blocks of different shapes, e.g., strips, blocks, tablets, discs, which release the compound out of the polymer matrix, membrane systems which hold the psyllid attractant within an impermeable container and release it through a measured permeable membrane, and combinations of the foregoing. Examples of other dispensing means are polymer laminates, polyvinyl chloride pellets, and microcapillaries.

Controlled release can also be achieved by a number of other methods such as, e.g., complexation of a psyllid attractant, slowly dissolving coatings, erosion, microbial action, or use of derivatives or new compounds of reduced solubility or volatility.

In aspects of this embodiment, a controlled-release device releases a psyllid attractant disclosed herein with substantially zero order release kinetics over a period of, e.g., about 7 days, about 15 days, about 30 days, about 45 days, about 60 days, about 75 days, or about 90 days. In other aspects of this embodiment, a controlled-release device releases a psyllid attractant disclosed herein with substantially zero order release kinetics over a period of, e.g., at least 7 days, at least 15 days, at least 30 days, at least 45 days, at least 60 days, at least 75 days, or at least 90 days. In other aspects of this embodiment, a controlled-release device releases a psyllid attractant disclosed herein with substantially zero order release kinetics over a period of between, e.g., about 7 days to about 30 days, about 15 days to about 45 days, about 30 days to about 60 days, about 45 days to about 75 days, or about 60 days to about 90 days.

In aspects of this embodiment, a controlled-release device releases a psyllid attractant disclosed herein with substantially first order release kinetics over a period of, e.g., about 7 days, about 15 days, about 30 days, about 45 days, about 60 days, about 75 days, or about 90 days. In other aspects of this embodiment, a controlled-release device releases a psyllid attractant disclosed herein with substantially first order release kinetics over a period of, e.g., at least 7 days, at least 15 days, at least 30 days, at least 45 days, at least 60 days, at least 75 days, or at least 90 days. In other aspects of this embodiment, a controlled-release device releases a psyllid attractant disclosed herein with substantially first order release kinetics over a period of between, e.g., about 7 days to about 30 days, about 15 days to about 45 days, about 30 days to about 60 days, about 45 days to about 75 days, or about 60 days to about 90 days.

Regardless of the method of application, the amount of a psyllid attractant disclosed herein used is an effective amount, i.e., an amount sufficient to attract or direct movement of psyllids to the source of psyllid attractant release. In aspects of this embodiment, a psyllid attractant disclosed herein is applied at a rate of, e.g., about 0.01 mg/m², about 0.025 mg/m², about 0.05 mg/m², about 0.075 mg/m², about 0.1 mg/m², about 0.25 mg/m², about 0.5 mg/m², about 0.75 mg/m², about 1 mg/m², about 2.5 mg/m², about 5 mg/m², about 7.5 mg/m², or about 10 mg/m². In other aspects of this embodiment, a psyllid attractant disclosed herein is applied at a rate of, e.g., at least 0.01 mg/m², at least 0.025 mg/m², at least 0.05 mg/m², at least 0.075 mg/m², at least 0.1 mg/m², at least 0.25 mg/m², at least 0.5 mg/m², at least 0.75 mg/m², at least 1 mg/m², at least 2.5 mg/m², at least 5 mg/m², at least 7.5 mg/m², or at least 10 mg/m². In yet other aspects of this embodiment, a psyllid attractant disclosed herein is applied at a rate of, between e.g., about 0.01 mg/m² to about 10 mg/m², about 0.01 mg/m² to about 1 mg/m², about 0.01 mg/m² to about 0.1 mg/m², about 0.05 mg/m² to about 10 mg/m², about 0.05 mg/m² to about 1 mg/m², about 0.05 mg/m² to about 0.1 mg/m², about 0.05 mg/m² to about 5 mg/m², or about 0.05 mg/m² to about 0.5 mg/m².

Aspects of the present specification disclose a method of capturing a psyllid using a psyllid attractant or composition disclosed herein. In an aspect of this embodiment, a method of capturing a psyllid uses a trap disclosed herein. In this disclosed method, psyllid attractant or composition disclosed herein attracts psyllids into the trap where the insects are captured.

Aspects of the present specification disclose a method of killing a psyllid using a psyllid attractant or composition disclosed herein. In an aspect of this embodiment, a method of capturing a psyllid uses a trap disclosed herein. In this disclosed method, psyllid attractant or composition disclosed herein attracts psyllids into the trap where the insects are killed.

Aspects of the present specification disclose a method of monitoring a psyllid population using a psyllid attractant or composition disclosed herein. In an aspect of this embodiment, a method of monitoring a psyllid population uses a trap disclosed herein. In this disclosed method, psyllid attractant or composition disclosed herein attracts psyllids into the trap where the insects are captured. Once captured, the number of captured psyllids can be determined simply by counting. Counted psyllids may then be analyzed using any suitable statistical analysis in order to determine the psyllid population. Such statistical analyses are well known to a person of ordinary skill in the art.

Aspects of the present specification disclose a use of a psyllid attractant or composition disclosed herein to attract psyllids. In one embodiment, a use of a psyllid attractant or composition disclosed herein is to attract psyllids into a trap disclosed herein in order to capture of kill the psyllids. In another aspect of this embodiment, a use of a psyllid attractant or composition disclosed herein to monitor a psyllid population. In yet another aspect of this embodiment, a use of a psyllid attractant or composition disclosed herein to attract psyllids to a particular location as a biocontrol for particular plant. In an aspect of this embodiment, a psyllid attractant or composition disclosed herein is used to attract broom psyllids in order to control the European broom (Cytisus scoparius), a pestilential invasive leguminous shrub. In another aspect of this embodiment, a psyllid attractant or composition disclosed herein is used to attract melaleuca psyllids in order to control the paperbark tree (Melaleuca quiquenervia). In yet another aspect of this embodiment, a psyllid attractant or composition disclosed herein is used to attract mesquite psyllids in order to control mesquite (Prosopis spp.).

Aspects of the present specification may also be described as follows:
1. A compound that substantially mimics the attractant chemosensory cues of a natural compound produced by a plant, wherein the compound attracts a psyllid.
2. A compound having a binding affinity for a psyllid OBP or SAP that is substantially the same as the binding affinity of a natural ligand for the psyllid OBP or SAP, wherein the compound attracts a psyllid.
3. The compound of embodiment 1 or 2, wherein the compound has a binding affinity for a psyllid OBP or SAP that is at least 75%, at least 80%, at least 85%, at least 90%, at least 95%, or at least 97% that of the binding affinity of the natural ligand for that psyllid OBP or SAP.
4. The compound of embodiment 1 or 2, wherein the compound has a dissociation equilibrium constant that is greater than the dissociation equilibrium constant of the natural ligand for that psyllid OBP or SAP by at least one-fold, at least two-fold, at least three-fold, at least four fold, at least five-fold, at least six-fold, at least seven-fold, at least eight-fold, at least nine-fold, at least 10-fold, at least 20-fold, at least 30-fold, at least 40-fold, at least 50-fold, at least 60-fold, at least 70-fold, at least 80-fold, at least 90-fold, at least 100-fold, at least 125-fold, at least 150-fold, at least 175-fold, or at least 200-fold.
5. The compound of embodiment 1 or 2, wherein the compound has an association rate constant of less than 1×10⁵ M⁻¹ s⁻¹, less than 1×10⁶ M⁻¹ s⁻¹, less than 1×10⁷ M⁻¹ s⁻¹, or less than 1×10⁸ M⁻¹ s⁻¹.
6. The compound of embodiment 1 or 2, wherein the compound has an association rate constant of more than 1×10⁵ M⁻¹ s⁻¹, more than 1×10⁶ M⁻¹ s⁻¹, more than 1×10⁷ M⁻¹ s⁻¹, or more than 1×10⁸ M⁻¹ s⁻¹.
7. The compound of embodiment 1 or 2, wherein the compound has a dissociation rate constant of less than 1×10⁻³ s⁻¹, less than 1×10⁻⁴ s⁻¹, or less than 1×10⁻⁵ s⁻¹.
8. The compound of embodiment 1 or 2, wherein the compound has a dissociation rate constant of more than 1×10⁻³ s⁻¹, more than 1×10⁻⁴ s⁻¹, or more than 1×10⁻⁵ s⁻¹.
9. The compound of embodiment 1 or 2, wherein the compound has an equilibrium dissociation constant of less than 0.500 nM, less than 0.450 nM, less than 0.400 nM, less than 0.350 nM, less than 0.300 nM, less than 0.250 nM, less than 0.200 nM, less than 0.150 nM, less than 0.100 nM, or less than 0.050 nM.
10. The compound of embodiment 1 or 2, wherein the compound has an equilibrium dissociation constant of more than 0.500 nM, more than 0.450 nM, more than 0.400 nM, more than 0.350 nM, more than 0.300 nM, more than 0.250 nM, more than 0.200 nM, more than 0.150 nM, more than 0.100 nM, or more than 0.050 nM.
11. The compound of embodiments 1-10, wherein the compound substantially mimics the attractant chemosensory cues of (R)-(+)-Limonene.
12. The compound of embodiments 1-10, wherein the compound has a binding affinity that is substantially the same as the binding affinity of (R)-(+)-Limonene for that psyllid OBP or SAP.
13. The compound of embodiment 11 or 12, wherein the compound has a binding affinity for a psyllid OBP or SAP that is at least 75%, at least 80%, at least 85%, at least 90%, at least 95%, or at least 97% that of the binding affinity of (R)-(+)-Limonene for that psyllid OBP or SAP.
14. The compound of embodiment 11, wherein the compound has a dissociation equilibrium constant that is greater than the dissociation equilibrium constant of (R)-(+)-Limonene for that psyllid OBP or SAP by at least one-fold, at least two-fold, at least three-fold, at least four fold, at least five-fold, at least six-fold, at least seven-fold, at least eight-fold, at least nine-fold, at least 10-fold, at least 20-fold, at least 30-fold, at least 40-fold, at least 50-fold, at least 60-fold, at least 70-fold, at least 80-fold, at least 90-fold, at least 100-fold, at least 125-fold, at least 150-fold, at least 175-fold, or at least 200-fold.
15. The compound of embodiments 1-10, wherein the compound substantially mimics the attractant chemosensory cues of Petitgrain.
16. The compound of embodiment 15, wherein the compound has a binding affinity that is substantially the same as the binding affinity of Petitgrain for that psyllid OBP or SAP.
17. The compound of embodiment 15 or 16, wherein the compound has a binding affinity for a psyllid OBP or SAP that is at least 75%, at least 80%, at least 85%, at least 90%, at least 95%, or at least 97% that of the binding affinity of Petitgrain for that psyllid OBP or SAP.
18. The compound of embodiment 15, wherein the compound has a dissociation equilibrium constant that is greater than the dissociation equilibrium constant of Petitgrain for that psyllid OBP or SAP by at least one-fold, at least two-fold, at least three-fold, at least four fold, at least five-fold, at least six-fold, at least seven-fold, at least eight-fold, at least nine-fold, at least 10-fold, at least 20-fold, at least 30-fold, at least 40-fold, at least 50-fold, at least 60-fold, at least 70-fold, at least 80-fold, at least 90-fold, at least 100-fold, at least 125-fold, at least 150-fold, at least 175-fold, or at least 200-fold.
19. The compound of embodiments 1-10, wherein the compound has a structure of formula I:

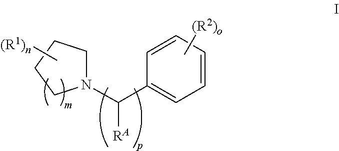

wherein R^{A} and R¹ are each independently R³, optionally substituted aryl, ═O, NR³R⁴, OR³, CN, NO₂, OCF₃, CF₃, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, OCF₃, CF₃, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl, OCF₃, or CF₃; m is 0, 1, 2, 3 or 4; n is 0, 1, 2, 3, 4, 5 or 6; p is 0, 1, 2, 3, 4, 5 or 6; and o is 0, 1, 2, 3, 4, or 5.
20. The compound of embodiment 19, wherein p is 1.
21. The compound of embodiment 19 or 20, wherein m is 2.
22. The compound of embodiment 19 or 20, wherein m is 3.
23. The compound of embodiments 19-21, wherein the compound has a structure of formula VI:

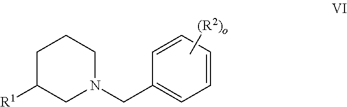

wherein R¹ is R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl, or CF₃; and o is 0, 1, 2, 3, 4, or 5.
24. The compound of embodiments 19-21, wherein the compound has a structure of formula VII:

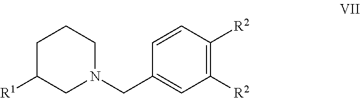

wherein R¹ is R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; and R³ and R⁴ are each independently H, optionally substituted alkyl, or CF₃.
25. The compound of embodiments 19-21, wherein the compound has a structure of formula VIII:

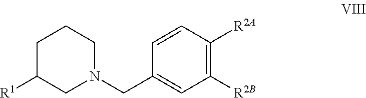

wherein R¹ is OH or ═O; and R^{2A} and R^{2B} are each independently H, Cl, Br, OH, CH₃, CH₂CH₃, or COOCH(CH₃)₂.
26. The compound of embodiment 19 or 20, wherein the compound has a structure of formula IX:

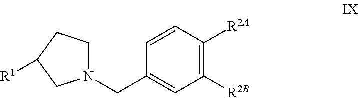

wherein R¹ is OH or ═O; and R^{2A} and R^{2B} are each independently H, Cl, Br, OH, CH₃, CH₂CH₃, or COOCH(CH₃)₂.
27. The compound of embodiments 1-10, wherein the compound has a structure of formula X:

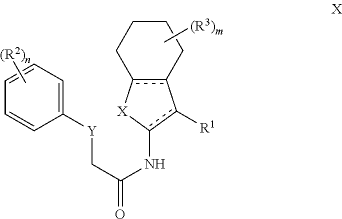

wherein a dashed line represent the presence or absence of a bond; R¹ and R³ are each independently R⁴, optionally substituted aryl, ═O, NR⁴R⁵, OR³, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; each R² is independently R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; R⁴ and R⁵ are each independently H, optionally substituted alkyl, or CF₃; m is 0, 1, 2, 3, 4, or 5; n is 0, 1, 2, 3, 4, or 5; X is CH₂, O, S, or NH; and Y is CH₂, O, S, or NH.
28. The compound of embodiment 27, wherein the compound has a structure of formula XI:

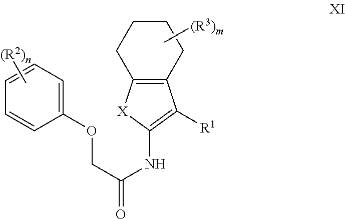

wherein each R³ is independently R⁴, optionally substituted aryl, ═O, NR⁴R⁵, OR³, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; R¹ and each R² is independently R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; R⁴ and R⁵ are each independently H, optionally substituted alkyl, or CF₃; m is 0, 1, 2, 3, 4, or 5; n is 0, 1, 2, 3, 4, or 5; and X is CH₂, O, S, or NH.
29. The compound of embodiment 27 or 28, wherein the compound has a structure of formula XII:

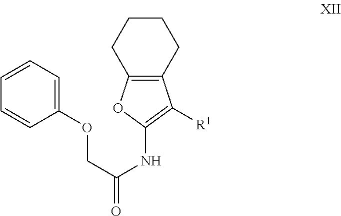

wherein R¹ is H, OH, Br, Cl, I, CH₃, NH₂, CN, NHCH₃, (CH₂)₂CH₃, CH₂Br, CH₂Cl, CH₃OH, COH, COOH, COOCH₃, COOCH₂CH₃, COOCH(CH₃)₂, OCH₃, OCH₂CH₃, or CONHCH₃.
30. The compound of embodiments 1-10, wherein the compound is 1-(3,4-dichlorobenzyl)-3-piperidinol, ethyl 2-[(phenoxyacetyl)amino]-4,5,6,7-tetrahydro-1-benzothiophene-3-carboxylate, 2-{[(3-chloro-4-fluorophenyl)amino]methylene}-5,5-dimethyl-1,3-cyclohexanedione, 1-(4-chlorobenzyl)-4-(4-nitrophenyl)piperazine, dimethyl 2-(1,2,2-trimethyl-3-thioxo-2,3-dihydro-4(1H)-quinolinylidene)-1,3-dithiole-4,5-dicarboxylate, N,N′-(2-nitro-1,3-propanediylidene)dicyclohexanamine, 2-[2-(3,5-dimethyl-1H-pyrazol-1-yl)-6-phenyl-4-pyrimidinyl]phenol, 4-methyl-2-[2-nitro-5-(1-pyrrolidinyl)phenyl]-1(2H)-phthalazinone, 2-[(4-methylphenyl)amino]naphthoquinone, 3-ethyl-4,7-dimethoxy-2-methyl-1,3-benzothiazol-3-ium iodide, 4-hydroxy-3-[2-(2-hydroxy-5-methoxyphenyl)-2,3-dihydro-1,5-benzothiazepin-4-yl]-6-methyl-2H-pyran-2-one, 2,4-dibromo-6-[(1,3-dioxo-1,3-dihydro-2H-inden-2-ylidene)methyl]phenyl acetate, ethyl 2-[(2-hydroxy-5-methoxybenzylidene)amino]-4,5-dimethyl-3-thiophenecarboxylate, 2-[2-(4-chlorophenyl)vinyl]-3-phenyl-4(3H)-quinazolinone, 7-methyl-2-oxo-1,3-benzoxathiol-5-yl benzenesulfonate, N-(5-benzylidene-4-oxo-2-thioxo-1,3-thiazolidin-3-yl)-2-methoxybenzamide, 2-methyl-N′-(2-oxo-1,2-dihydro-3H-indol-3-ylidene)benzohydrazide, 5-(4-aminophenoxy)-2-phenyl-1H-isoindole-1,3(2H)-dione, 3-chloro-6-fluoro-N-(4-phenyl-1,3-thiazol-2-yl)-1-benzothiophene-2-carboxamide, N′-(2-hydroxy-5-methoxybenzylidene)-4-(2-oxo-1-pyrrolidinyl)benzohydrazide, 2-(2,5-dichlorophenyl)-1,3-benzoxazol-5-amine, 2-hydroxy-N′-[(2-hydroxy-1-naphthyl)methylene]-2-phenylpropanohydrazide, 2-(3-bromo-2-hydroxy-5-nitrobenzylidene)-1-benzothiophen-3(2H)-one, 4-[2-(1,3-benzodioxol-5-yl)vinyl]quinoline, ethyl 2-amino-4-(4-bromophenyl)-5-oxo-4H,5H-pyrano[3,2-c]chromene-3-carboxylate, (5-chloro-2-methylphenyl)[3-(4-nitrophenyl)-2-propen-1-ylidene]amine, 1-{[(4′-nitro-4-biphenyl)imino]methyl}-2-naphthol, N-[4-chloro-3-(trifluoromethyl)phenyl]-7-nitro-2,1,3-benzoxadiazol-4-amine, 3-allyl-2-(benzylthio)-5-ethyl-6-hydroxy-4(3H)-pyrimidinone, 1H-indole-2-carbaldehyde (1H-indol-2-ylmethylene)hydrazone, 1-(2-bromobenzoyl)-4-phenylpiperazine, (4-bromobenzylidene)(7-nitro-9H-fluoren-2-yl)amine, 3-(3-bromophenyl)-5-(2-furylmethylene)-2-thioxo-1,3-thiazolidin-4-one, 2,4-dibromo-6-[(5-methyl-1,3,4-thiadiazol-2-yl)amino]phenol, 5-{3-[4-(dimethylamino)phenyl]-2-propen-1-ylidene}-2-thioxo-1,3-thiazolidin-4-one, 6-[2-(2,4-dichloro-5-nitrophenyl)vinyl]-4-(trifluoromethyl)-2(1H)-pyrimidinone, 5-{[(3,5-dimethoxyphenyl)amino]methylene}-1-(3-methylphenyl)-2-thioxodihydro-4,6(1H,5H)-pyrimidinedione, N-[3-(3-bromophenyl)-2-propen-1-ylidene]-4-(4-morpholinyl)aniline, 5-[4-(diethylamino)benzylidene]-2-(4-morpholinyl)-1,3-thiazol-4(5H)-one, 3-bromo-N′-(3-oxo-3-phenyl-1-propen-1-yl)benzohydrazide, 1-{[5-(4-nitrophenyl)-2-furyl]methyl}-4-phenylpiperazine, 1-(4-ethylcyclohexyl)-4-(4-nitrophenyl)piperazine, 1-[3-(2-nitrophenyl)-2-propen-1-yl]-4-(3-phenyl-2-propen-1-yl)piperazine, 1-(4-nitrophenyl)-4-(4-pyridinylmethyl)piperazine, 1-(4-nitrophenyl)-4-(3-phenyl-2-propen-1-yl)piperazine, 6,8-dichloro-3-{[4-(2-methoxyphenyl)-1-piperazinyl]methyl}-4H-chromen-4-one, 1-(bicyclo[2.2.1]hept-5-en-2-ylmethyl)-4-(4-nitrophenyl)piperazine, 1-[(5-methyl-2-thienyl)methyl]-4-(4-nitrophenyl)piperazine, N,N-diethyl-4-[(phenylimino)methyl]aniline, 1-[4-(benzyloxy)phenyl]-3-methyl-1H-pyrrole-2,5-dione, 1-(4-nitrophenyl)-1H-indole, 4-{[(3-bromophenyl)imino]methyl}-1,3-benzenediol, 1,2-dimethyl-3-[2-(4-nitrophenyl)-2-oxoethyl]-1H-3,1-benzimidazol-3-ium bromide, 3-methyl-2-[4-oxo-5-(2-oxo-1,2-dihydro-3H-indol-3-ylidene)-2-thioxo-1,3-thiazolidin-3-yl]butanoic acid, 4-bromo-N′-(2-oxo-1,2-dihydro-3H-indol-3-ylidene)benzohydrazide, N-[2-(2-naphthyl)-1,3-dioxo-2,3-dihydro-1H-isoindol-4-yl]acetamide, N′-(1,7-dimethyl-2-oxo-1,2-dihydro-3H-indol-3-ylidene)-2-(4-methoxyphenoxy)acetohydrazide, 5-[(2-ethoxy-1-naphthyl) methylene]-2,4,6(1H,3H,5H)-pyrimidinetrione, 2-(2,6-dibromo-4-methylphenoxy)-N′-(2-oxo-1,2-dihydro-3H-indol-3-ylidene)acetohydrazide, 4-{4-[(4-nitrophenyl)thio]phenyl}-4-azatricyclo[5.2.1.0˜2,6˜]dec-8-ene-3,5-dione, 1-(4-bromophenyl)-5-[4-(dimethylamino)benzylidene]-2,4,6(1H,3H,5H)-pyrimidinetrione, 1-(2-methoxyphenyl)-5-(3-phenyl-2-propen-1-ylidene)-2,4,6(1H,3H,5H)-pyrimidinetrione, N-benzyl-7-chloro-4-nitro-2,1,3-benzoxadiazol-5-amine, 2-[3-(4-hydroxyphenyl)acryloyl]-1H-indene-1,3(2H)-dione, N′-(3,5-dibromo-2-hydroxybenzylidene)-4-[(4-methylphenyl)amino]butanohydrazide, 3-amino-6-cyclopropyl-4-phenylthieno[2,3-b]pyridine-2-carboxylic acid, 1-(dichloroacetyl)-4-(4-nitrophenyl)piperazine, 1-(5-bromo-2-methoxybenzyl)-4-(4-nitrophenyl)piperazine, 3-(3-bromophenyl)-5-(4-hydroxy-3-methoxybenzylidene)-2-thioxo-1,3-thiazolidin-4-one, 11-chloro-12-nitro-2-phenyl-2,4,5,6,7,8-hexahydro-3H-azepino[1,2-a]pyrazolo[4,3-c]quinolin-3-one, 4-benzyl-1-[(5-nitro-2-thienyl)methyl]piperidine, 3-[(benzylimino)methyl]-1,2-benzenediol, 2-[(6-nitro-1,3-benzodioxol-5-yl)methyl]-2,3,4,9-tetrahydro-1H-beta-carboline, 4-[4-(dimethylamino)benzylidene]-2-(3-nitrophenyl)-1,3-oxazol-5(4H)-one, 4-(2-oxo-1-pyrrolidinyl)-N′-[1-(2-pyridinyl)ethylidene]benzohydrazide, 2-[4-(methylthio)benzyl]-2,3,4,9-tetrahydro-1H-beta-carboline, 4-(dimethylamino)benzaldehyde (2-nitrophenyl)hydrazone, (4-bromophenyl)(5-methoxy-2-methyl-1-benzofuran-3-yl)methanone, 4-[(cyclohexylamino)methylene]-5-methyl-2-phenyl-2,4-dihydro-3H-pyrazole-3-thione, 4-bromo-2-{[(5-tert-butyl-2-hydroxyphenyl)imino]methyl}-6-ethoxyphenol, N′-(4-chlorobenzylidene)-1-methyl-5-phenyl-1H-pyrazole-3-carbohydrazide, N′-{[2-(4-bromophenyl)-3-indolizinyl]methylene}isonicotinohydrazide, ethyl 2-amino-4-(3,4-dichlorophenyl)-5-oxo-4H,5H-pyrano[3,2-c]chromene-3-carboxylate, 1H-indene-1,2,3-trione 2-(phenylhydrazone), 2-[(1,3-benzothiazol-2-ylamino)methylene]-1-benzothiophen-3(2H)-one, N-[2-[4-(dimethylamino)phenyl]-1-(1-piperazinylcarbonyl)vinyl]benzamide, 5-(2-hydroxy-3-methoxybenzylidene)-2-thioxo-1,3-thiazolidin-4-one, 1-[(4-chloro-3-nitrophenyl)sulfonyl]-2-methylpiperidine, 2-(1H-benzimidazol-2-ylthio)-N-(4-methylphenyl)acetamide, 3-[2-(4-chlorophenyl)-1,3-thiazol-4-yl]-7-hydroxy-2H-chromen-2-one, 4,4′-[1,4-phenylenebis(nitrilomethylylidene)]diphenol, 3-chloro-1-(4-chlorophenyl)-4-(dimethylamino)-1H-pyrrole-2,5-dione, 1-(4-bromophenyl)-3-chloro-4-(1-pyrrolidinyl)-1H-pyrrole-2,5-dione, N-{5-[4-(dimethylamino)benzylidene]-4-oxo-2-thioxo-1,3-thiazolidin-3-yl}-2-hydroxybenzamide, 5-{[5-(4-bromophenyl)-2-furyl]methylene}-1,3-thiazolidine-2,4-dione, 2,4,5-trimethylbenzaldehyde (4-amino-5-ethyl-4H-1,2,4-triazol-3-yl)hydrazone, 1-(2-chlorophenyl)-5-{[(3-chlorophenyl)amino]methylene}-2-thioxodihydro-4,6(1H,5H)-pyrimidinedione, 9-(3,5-dichloro-4-hydroxyphenyl)-3,3,6,6-tetramethyl-3,4,5,6,7,9-hexahydro-1H-xanthene-1,8(2H)-dione, 5-{3-methoxy-4-[2-oxo-2-(1-piperidinyl)ethoxy]benzylidene}-2-thioxo-1,3-thiazolidin-4-one, 3-(4-biphenylyl)-5,6-dihydroimidazo[2,1-b][1,3]thiazole hydrobromide, 5-(4-ethylbenzylidene)-2-thioxo-1,3-thiazolidin-4-one, 4-bromo-N′-(3-oxo-3-phenyl-1-propen-1-yl)benzohydrazide, 1-(2-fluorophenyl)-4-{[5-(2-nitrophenyl)-2-furyl]methyl}piperazine, 1-[(5-methyl-2-furyl)methyl]-4-(4-nitrophenyl)piperazine, N-benzyl-N-(4-nitrobenzyl)ethanamine, 6,8-dichloro-3-[(4-methyl-1,4-diazepan-1-yl)methyl]-4H-chromen-4-one, 3-[(4-acetyl-1-piperazinyl)methyl]-6,8-dichloro-4H-chromen-4-one, 2-(4-butoxybenzyl)-6,7-dimethoxy-1,2,3,4-tetrahydroisoquinoline, 2-{[butyl(methyl)amino]methyl}-4-nitrophenol, 6-chloro-3-{[2-(hydroxymethyl)-1-piperidinyl]methyl}-4H-chromen-4-one, 6,8-dichloro-3-(4-thiomorpholinylmethyl)-4H-chromen-4-one, 1-[(4-phenyl-1-piperazinyl)methyl]-2-naphthol, or 2-(1-azepanylmethyl)-6-ethoxyphenol.
31. A composition comprising one or more of the compounds of embodiments 1-30.
32. The composition of embodiment 31, wherein the composition optionally comprises an adhesive, a solvent, a wetting agent, an emulsifying agent, a carrier, a diluent, or a dispersing agent.
33. The composition of embodiment 31 or 32, wherein the composition optionally comprises an insecticide.
34. The composition of embodiment 33, wherein the insecticide includes Aldrin, Chlordane, Chlordecone, DDT, Dieldrin, Endosulfan, Endrin, Heptachlor, Hexachlorobenzene, Lindane (gamma-hexachlorocyclohexane), Methoxychlor, Mirex, Pentachlorophenol, TDE, an organophosphate, a carbamate, a pyrethroid, a neonicotinoid, or any combination thereof.
35. The composition of embodiment 34, wherein the organophosphate is Acephate, Azinphos-methyl, Bensulide, Chlorethoxyfos, Chlorpyrifos, Chlorpyriphos-methyl, Diazinon, Dichlorvos (DDVP), Dicrotophos, Dimethoate, Disulfoton, Ethoprop, Fenamiphos, Fenitrothion, Fenthion, Fosthiazate, Malathion, Methamidophos, Methidathion, Mevinphos, Monocrotophos, Naled, Omethoate, Oxydemeton-methyl, Parathion, Parathion-methyl, Phorate, Phosalone, Phosmet, Phostebupirim, Phoxim, Pirimiphos-methyl, Profenofos, Terbufos, Tetrachlorvinphos, Tribufos, Trichlorfon, or any combination thereof.
36. The composition of embodiment 34, wherein the carbamate is Aldicarb, Bendiocarb, Carbofuran, Carbaryl, Dioxacarb, Fenobucarb, Fenoxycarb, Isoprocarb, Methomyl, 2-(1-Methylpropyl)phenyl methylcarbamate, or any combination thereof.
37. The composition of embodiment 34, wherein the pyrethroid is Allethrin, Bifenthrin, Cyhalothrin, A-Cyhalothrin, Cypermethrin, Cyfluthrin, Deltamethrin, Etofenprox, Fenvalerate, Permethrin, Phenothrin, Prallethrin, Resmethrin, Tetramethrin, Tralomethrin, Transfluthrin, or any combination thereof.
38. The composition of embodiment 34, wherein the neonicotinoid is Acetamiprid, Clothianidin, Imidacloprid, Nitenpyram, Nithiazine, Thiacloprid, Thiamethoxam, or any combination thereof.
39. The composition of embodiments 31-38, wherein the composition further comprises a compatible matrix.
40. The composition of embodiments 31-39, wherein the composition is a controlled-release device.
41. The composition of embodiment 40, wherein the controlled-release device is a reservoir device or a monolithic device.
42. The composition of embodiments 31-41, wherein the composition is a liquid composition, a semi-solid composition, or a solid composition.
43. A device comprising one or more of the compounds of embodiments 1-30 or one or more compositions of embodiments 31-42.
44. The device of embodiment 43, wherein the device is designed to attract and/or capture psyllids.
45. The device of embodiment 43 or 44, wherein the device captures psyllids in a manner designed to keep the captured psyllids alive.
46. The device of embodiment 43 or 44, wherein the device captures psyllids in a manner designed to kill the captured psyllids.
47. The device of embodiments 43-46, wherein the device further comprises an adhesive.
48. The device of embodiments 43-47, wherein the device further comprises an insecticide.
49. The device of embodiments 43-48, wherein the device is a blunder trap.
50. The device of embodiments 43-48, wherein the device further comprises a container including a chamber operably-connected to an entrance.
51. The device of embodiment 50, wherein the entrance allows psyllids to freely ingress or egress from the chamber.
52. The device of embodiment 50, wherein the entrance is designed to hinder or prevent psyllids from leaving the chamber once entered.
53. A method of capturing psyllids, the method comprising using one or more of the compounds of embodiments 1-30 or one or more compositions of embodiments 31-42 to attract the psyllids to a device according to embodiments 43-52, thereby capturing the psyllids.
54. A method of killing psyllids, the method comprising using one or more of the compounds of embodiments 1-30 or one or more compositions of embodiments 31-42 to attract the psyllids to a device according to embodiments 43-52, thereby killing the psyllids.
55. A method of monitoring a psyllid population, the method comprising a) using one or more of the compounds of embodiments 1-30 or one or more compositions of embodiments 31-42 to attract psyllids to a device according to embodiments 43-52, thereby capturing the psyllids; b) counting the psyllids to determine a number of captured psyllids; and c) performing a statistical analysis on the number of captured psyllids in order to determine the psyllid population, thereby monitoring the psyllid population.
56. A compound, composition, device or method of embodiments 1-55, wherein the psyllid is a species from the family Psyllidae.

### EXAMPLES

The following non-limiting examples are provided for illustrative purposes only in order to facilitate a more complete understanding of the disclosed subject matter. These examples should not be construed to limit any of the embodiments described in the present specification, including those pertaining to the compounds, compositions, traps, methods or uses of psyllid attractants.

### Example 1

### Isolation of Candidate Compounds Using a Protein-Binding Assay

To identify a psyllid attractant disclosed herein, candidate compounds were screened based upon the ability of that compound to bind psyllid OBP1 (SEQ ID NO: 1), SAP1 (SEQ ID NO: 2), SAP2 (SEQ ID NO: 3), SAP3 (SEQ ID NO: 4), and/or SAP4 (SEQ ID NO: 5). By utilizing a fluorescent-quench, ligand-protein-binding assay, 30,000 compounds from the DIVERSET® library (ChemBridge Corp., San Diego, Calif.), a chemical library, were screened and over 150 candidate compounds were isolated that bound to each of the evaluated psyllid chemosensory proteins. An in silica structural analysis of candidate compound was then used to group them into families according to structure motifs and chemical composition in order to prioritize compounds for behavioral testing.

### Example 2

### Testing of Candidate Compounds Using a Leaf Probing Assay

To determine whether a living psyllid can recognize a candidate compound, these compounds were tested using a leaf-probing assay, a “no choice” test that measures probing activity of psyllids.

To conduct a leaf probing assay, a sachet is constructed by stretching PARAFILM® M over the opening of a 52 mm² plastic Petri dish. The enclosed dish, which functions as a reservoir, contains a 100 mM sucrose solution to provide the psyllids with water and nutrients during the 24 hour long testing period. Each candidate compound, as well as positive and negative control compounds, is mixed with a biologically inert and UV resistant matrix material called SPLAT (Specialized Pheromone & Lure Application Technology; ISCA Technologies, Riverside Calif.). A thin line of this mixture was applied to the surface of the PARAFILM® M in the shape of an X using a syringe in order to mimic the “midribs” of a leaf. The dish-membrane assembly was placed inside a larger plastic Petri dish that serves to confine the psyllids. Immediately prior to the start of each trial, the psyllids are anaesthetized by chilling and five individuals were placed onto the membrane. The cover of the large Petri dish is replaced and the dishes were placed in an incubator kept at 28° C. (±1° C.) with a 14:10 (L:D) photoperiod. After 24 hrs, the dishes were removed from the incubator and the numbers of surviving psyllids counted. Post-mortem examinations are conducted to determine the number of males and females in each dish.

To visualize the psyllid probing, membrane-dish assemblies were submerged in Coomassie blue solution for 5 minutes to stain the salivary sheaths left in the SPLAT when the psyllids probed the wax line. Following rinsing in distilled water, the wax lines were air dried and the number of probing holes determined in each treatment. To count the number of probing holes, two lines, each 5 mm long, were drawn with a fine point pen on the membrane adjacent to each arm of each line, so that a total of eight sections are sampled per replicate. The sections were examined with a dissection microscope and all of the probing holes counted in each section.

Initially two concentrations of a candidate compound were tested: 1) High: 5 mg compound in 10 mL of SPLAT; and 2) Low: 1.7 mg compounds in 10 mL of SPLAT. In addition, compounds that are positive at the low concentration will be tested at 1:20 and 1:100. The results indicate that Compound 1 significantly increased leaf probing when compared to both the blank negative control and the positive control (Table 1).

TABLE 1
Results of Leaf Probing Assay

| | | | Compound 1 | Compound 1 |
|---|---|---|---|---|
| | Blank | Positive Control | [Low] | [High] |
| N | 30 | 30 | 30 | 30 |
| Mean | 6.2 ± 0.8 | 11.1 ± 1.2 | 39.7 ± 5.3 | 13.5 ± 1.3 |

### Example 3

### Testing of Candidate Compounds Using a Modified Olfactory Assay

To differentiate between candidate compounds that act as olfactory attractants as oppose to gustatory attractants, a modified olfactory assay was performed on candidate compounds testing positive in the leaf probing assay.

To construct an olfactometer, a glass tube was covered at one end with a perforated aluminum cover shaped to form a cup. A synthetic tubing was connected at the opposite end of the glass tube which was in turn connected via an airline system to an air pump (KNF Laboratories, Trenton, N.J.) and a 1,000-m1 Erlenmeyer flask with 500 mL of distilled water provided airflow and humidity to the system. The olfactometer was oriented horizontally and situated 30 cm below fluorescent lights.

To conduct a modified olfactory assay, individual psyllids were anaesthetized by chilling (60 seconds) and then placed into the center of the perforated cup. Psyllids were obtained from an indoor colony reared on orange jasmine and curry leaf. Individuals were starved from 1 to 3 hrs prior to testing. Candidate compounds were dissolved in SPLAT, added to a small square of filter paper and placed within a glass flask connected to the airline system. The volatilized compounded was then passed through the tube and into the glass tube and perforated cup. Each test ran for four minutes, psyllids that left the cup prior to 15 seconds were not included because of the strong possibility that they were engaged in an escape response. Psyllids were exposed to only a single odorant. Four treatments were tested: 1) Blank air (negative control), 2) Blank SPLAT, 3) Freshly-cut orange jasmine flushing foliage (positive control), and 4) Candidate compound mixed in SPLAT (6 mg compound/6 mL SPLAT). Odor sources were placed into an in-line glass container. Three fresh sprigs of orange jasmine were used in the foliage treatment and room air was used in the air blank treatment. The blank SPLAT and Titan mixed in SPLAT treatments were applied with an artist's paintbrush to a microscope slide placed in the odor source container. Air was supplied to each line with a small vacuum pump at a flow rate of 230 mL/min. The response variable was retention time in the cup; i.e., the amount of time it to took a psyllid to move from the center of the cup to the rim. The behavioral basis of the response is that if an odorant is stimulatory, then perception of that odor will induce localized searching behavior; i.e., retention times are lower when psyllid are presented with neutral odors and longer when presented with odors that are attractive.

The results show that Compound 1 is attractive to psyllids to a statistically significant degree when compared to blank air (P=0.0077) and blank SPLAT (P=0.0349) controls and just as attractive as compared to orange jasmine flushing foliage positive control (Table 2).

TABLE 2
Results of Olfactometry Study

| | Males | | | | Females | | | |
|---|---|---|---|---|---|---|---|---|
| | | Blank | | Compound | | Blank | | Compound |
| | Air | SPLAT | Foliage | 1 | Air | SPLAT | Foliage | 1 |
| N | 70 | 68 | 82 | 83 | 24 | 31 | 35 | 38 |
| Mean | 127 ± | 114 ± | 84 ± | 76 ± | 115 ± | 92 ± | 98 ± | 110 ± |
| | 8 | 8 | 6 | 7 | 14 | 13 | 6 | 8 |

### Example 4

### Greenhouse Trials Using Candidate Compounds

This example illustrates how to test a psyllid attractant disclosed herein for its ability to attract psyllids to a trap comprising the attractant.

To conduct a green house trial, sticky traps will be deployed in an environmentally-controlled greenhouse house comprising healthy citrus trees to simulate an orchard. These sticky traps will include a composition comprising 1 mg of a candidate compound and 10 mL of a biologically inert, UV resistant matrix material (SPLAT). Control traps will contain SPLAT alone as a negative control and SPLAT plus a natural citrus scent as a positive control. Roughly 200 psyllids per trial will be used and experiments will be conducted to compare the efficacy of sticky traps with a control compared to sticky traps with a candidate compound in terms of attracting live psyllids. Candidate compounds resulting in the best measured attraction will be refined and further evaluated in small-scale field trials.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, e.g., the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1-56. (canceled)

57. A compound having a structure of formula I: 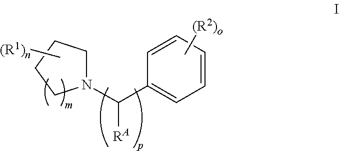
wherein R^{A} and R¹ are each independently R³, optionally substituted aryl, ═O, NR³R⁴, OR³, CN, NO₂, OCF₃, CF₃, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, OCF₃, CF₃, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl, OCF₃, or CF₃; m is 0, 1, 2, 3 or 4; n is 0, 1, 2, 3, 4, 5 or 6; p is 0, 1, 2, 3, 4, 5 or 6; and o is 0, 1, 2, 3, 4, or 5.

58. The compound of claim 57, wherein the compound has a structure of formula VI: 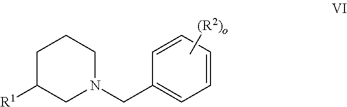
wherein R¹ is R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; R³ and R⁴ are each independently H, optionally substituted alkyl, or CF₃; and o is 0, 1, 2, 3, 4, or 5.

59. The compound of claim 58, wherein the compound has a structure of formula VII: 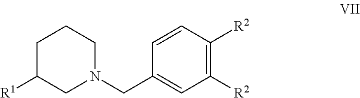
wherein R¹ is R³, optionally substituted aryl, ═O, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; each R² is independently R³, optionally substituted aryl, NR³R⁴, OR³, CN, NO₂, Br, Cl, F, SR³, SOR³, SO₂R³, OCOR³, CO₂R³, COR³, CONR³R⁴, NR³COR⁴, or CSNR³R⁴; and R³ and R⁴ are each independently H, optionally substituted alkyl, or CF₃.

60. The compound of claim 59, wherein the compound has a structure of formula VIII: 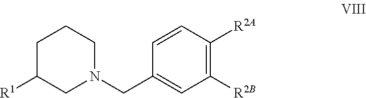
wherein R¹ is OH or ═O; and R^{2A} and R^{2B} are each independently H, Cl, Br, OH, CH₃, CH₂CH₃, or COOCH(CH₃)₂.

61. The compound of claim 57, wherein the compound has a structure of formula IX: 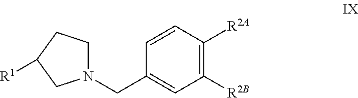
wherein R¹ is OH or ═O; and R^{2A} and R^{2B} are each independently H, Cl, Br, OH, CH₃, CH₂CH₃, or COOCH(CH₃)₂.

62. The compound of claim 57, wherein the compound has a binding affinity for a psyllid OBP or SAP having an equilibrium dissociation constant of less than 0.500 nM, less than 0.450 nM, less than 0.400 nM, less than 0.350 nM, less than 0.300 nM, less than 0.250 nM, less than 0.200 nM, less than 0.150 nM, less than 0.100 nM, or less than 0.050 nM.

63. A composition comprising one or more of the compounds as defined in claim 57.

64. The composition of claim 57, wherein the composition is a liquid composition, a semi-solid composition, or a solid composition.

65. A device comprising one or more of the compounds as defined in claim 57.

66. A method of capturing psyllids, the method comprising using one or more of the compounds as defined in claim 57 to attract the psyllids to a device, thereby capturing the psyllids.

67. A method of killing psyllids, the method comprising using one or more of the compounds as defined in claim 57 to attract the psyllids to a device, thereby killing the psyllids.

68. A method of monitoring a psyllid population, the method comprising a) using one or more of the compounds as defined in claim 57 to attract psyllids to a device, thereby capturing the psyllids; b) counting the psyllids to determine a number of captured psyllids; and c) performing a statistical analysis on the number of captured psyllids in order to determine the psyllid population, thereby monitoring the psyllid population.

69. A compound having a structure of formula X: 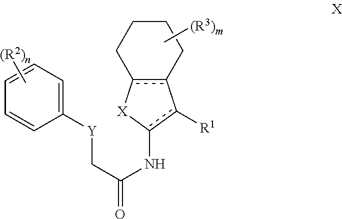
wherein a dashed line represent the presence or absence of a bond; R¹ and R³ are each independently R⁴, optionally substituted aryl, ═O, NR⁴R⁵, OR³, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; each R² is independently R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; R⁴ and R⁵ are each independently H, optionally substituted alkyl, or CF₃; m is 0, 1, 2, 3, 4, or 5; n is 0, 1, 2, 3, 4, or 5; X is CH₂, O, S, or NH; and Y is CH₂, O, S, or NH.

70. The compound of claim 69, wherein the compound has a structure of formula XI: 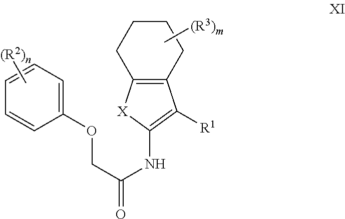
wherein each R³ is independently R⁴, optionally substituted aryl, ═O, NR⁴R⁵, OR³, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; R¹ and each R² is independently R⁴, optionally substituted aryl, NR⁴R⁵, OR⁴, CN, NO₂, Br, Cl, F, SR⁴, SOR⁴, SO₂R⁴, OCOR⁴, CO₂R⁴, COR⁴, CONR⁴R⁵, NR⁴COR⁵, or CSNR⁴R⁵; R⁴ and R⁵ are each independently H, optionally substituted alkyl, or CF₃; m is 0, 1, 2, 3, 4, or 5; n is 0, 1, 2, 3, 4, or 5; and X is CH₂, O, S, or NH.

71. The compound of claim 70, wherein the compound has a structure of formula XII: 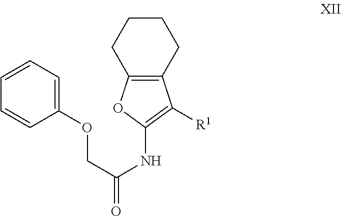
wherein R¹ is H, OH, Br, Cl, I, CH₃, NH₂, CN, NHCH₃, (CH₂)₂CH₃, CH₂Br, CH₂Cl, CH₃OH, COH, COOH, COOCH₃, COOCH₂CH₃, COOCH(CH₃)₂, OCH₃, OCH₂CH₃, or CONHCH₃.

72. A composition comprising one or more of the compounds as defined in claim 69.

73. A device comprising one or more of the compounds as defined in claim 69.

74. A method of capturing psyllids, the method comprising using one or more of the compounds as defined in claim 69 to attract the psyllids to a device, thereby capturing the psyllids.

75. A method of killing psyllids, the method comprising using one or more of the compounds as defined in claim 69 to attract the psyllids to a device, thereby killing the psyllids.

76. A method of monitoring a psyllid population, the method comprising a) using one or more of the compounds as defined in claim 69 to attract psyllids to a device, thereby capturing the psyllids; b) counting the psyllids to determine a number of captured psyllids; and c) performing a statistical analysis on the number of captured psyllids in order to determine the psyllid population, thereby monitoring the psyllid population.
